(19) United States
(12) Patent Application Publication
(10) Pub. No.: US 2018/0272147 A1
(43) Pub. Date: Sep. 27, 2018

(54) WEARABLE DEFIBRILLATOR INTEGRATED WITH REMOTE ISCHEMIC CONDITIONING PROTOCOL

(71) Applicant: ZOLL Medical Corporation, Chelmsford, MA (US)

(72) Inventors: Gary A. Freeman, Waltham, MA (US); Gregory R. Frank, Mt. Lebanon, PA (US)

(21) Appl. No.: 15/922,958

(22) Filed: Mar. 16, 2018

Related U.S. Application Data
(60) Provisional application No. 62/474,176, filed on Mar. 21, 2017.

(51) Int. Cl. A61N 1/39; A61F 7/00; A61H 9/00; A61H 31/00

(52) U.S. Cl. CPC A61N 1/3987; A61F 7/0085; A61B 5/0225; A61H 9/0092; A61H 31/006; A61N 1/3904

(57) ABSTRACT

A wearable medical treatment system for medical treatment of a patient is described. An example of the system includes sensors configured to be externally positioned on the patient, the sensors including cardiac sensing electrodes configured to provide a signal indicative of the patient's cardiac information, defibrillation electrodes configured to be externally positioned on the patient and to deliver a defibrillation shock, a cuff configured to contract about a patient's limb, and a controller communicatively coupled to the sensors, the defibrillation electrodes, and the cuff. The controller is configured to receive the signal indicative of the patient's cardiac information, determine the patient's cardiac information, control delivery of the defibrillation shock by the defibrillation electrodes based at least in part on the patient's cardiac information, and control contraction of the cuff based according to a remote ischemic conditioning (RIC) protocol and based at least in part on the patient's cardiac information.

### CROSS-REFERENCE TO RELATED APPLICATIONS

This application claims priority under 35 U.S.C. § 119 to U.S. Patent Application No. 62/474,176 filed on Mar. 21, 2017. All subject matter set forth in the above referenced application is hereby incorporated by reference in its entirety into the present application as if fully set forth herein.

### BACKGROUND

An external and wearable cardiac monitoring and medical treatment system can provide therapeutic and/or resuscitative care to a patient at risk for adverse cardiac events. The ZOLL® LifeVest® wearable cardioverter defibrillator available from ZOLL® Medical Corporation is an example of such a system. The external monitoring and treatment system may include one or more medical devices that are at least partially disposed outside of the patient's body (i.e., that are not completely implanted within the patient's body). The wearable monitoring and treatment system is capable of and designed for continuous use by the patient as the patient goes about his or her daily routine. Continuous use includes substantially continuous use. For example, the wearable system may be continuously used, except for sporadic periods during which the use temporarily ceases (e.g., while the patient bathes, while the patient is refit with a new and/or a different garment, while the battery is charged and/or changed, while the garment is laundered, etc.). The patient may wear the wearable system for an extended time period (e.g., one or more days, one or more weeks, one or more months, or one or more years). The patient may wear the wearable system, for example, in an in-patient setting, in an out-patient setting, and/or in a specialized setting such as, for example, a combat zone or inside an emergency vehicle.

### SUMMARY

An example of a wearable medical treatment system for medical treatment of a patient according to the disclosure includes a plurality of sensors configured to be externally positioned on the patient, the plurality of sensors including at least one cardiac sensing electrode configured to provide one or more signals indicative of cardiac information of the patient, at least one defibrillation electrode configured to be externally positioned on the patient and to deliver a defibrillation shock to the patient, a cuff configured to contract about a limb of the patient, and a controller communicatively coupled to the plurality of sensors, the at least one defibrillation electrode, and the cuff. The controller is configured to receive the one or more signals indicative of the cardiac information of the patient, determine the cardiac information of the patient, control delivery of the defibrillation shock by the at least one defibrillation electrode based at least in part on the cardiac information of the patient, and control contraction of the cuff based according to a remote ischemic conditioning (RIC) protocol and based at least in part on the cardiac information of the patient.

Implementation of such a system may include one or more of the following features. The RIC protocol may be determined based at least in part on the cardiac information of the patient. The controller may be configured to determine a cardiac event risk score associated with a potential adverse cardiac event of the patient based at least in part on the cardiac information of the patient, and implement the RIC protocol based on the cardiac event risk score. The RIC protocol may be predetermined. The wearable medical treatment may include a user interface communicatively coupled to the controller and configured to capture input from the patient. The controller may be configured to control contraction of the cuff based on the input from the patient. The controller may be configured to determine the RIC protocol based on the input from the patient. The RIC protocol may include a plurality of sequential treatment cycles and each cycle may include a cuff contraction phase during which the controller causes the cuff to contract about the limb of the patient to a set point pressure above a systolic pressure of the patient to occlude blood flow through the limb, an ischemic phase during which the controller causes the cuff to remain contracted about the limb of the patient at the set point pressure for an ischemic phase duration, a cuff release phase during which the controller causes the cuff to relax to allow the blood flow through the limb, and a reperfusion phase during which the controller causes the cuff to remain relaxed to allow the blood flow through the limb for a reperfusion phase duration. The ischemic phase duration may be between 0.5 and 30 minutes. The reperfusion phase duration may be between 0.5 and 30 minutes. The RIC protocol may include a time interval at which to implement the plurality of sequential treatment cycles. The time interval may be a number of times per week. The time interval may be between 1 and 7 times per week. The time interval may be a number of times per day. The time interval may be between 1 and 5 times per day. The wearable medical treatment system may include a gas pressure source, a gas flow regulator coupled to the gas pressure source and the controller, and a pneumatic distribution system coupled to the gas flow regulator. The cuff may include a pneumatically inflatable bladder removably connected to the pneumatic distribution system. The controller may be configured to adjust a gas pressure in the pneumatically inflatable bladder via control of the gas flow regulator. The cuff may include a connection port and the pneumatic distribution system may include a gas flow tube configured to removably connect to the connection port. The pneumatically inflatable bladder may be confined to a portion of the cuff configured to be proximate to a surface of an arm of the patient that approximately faces the torso of the patient. The wearable medical treatment system may include a garment that may be configured to be worn about the torso of the patient. The pneumatic distribution system may be integrated into the garment. At least a portion of the garment may include pneumatically inflatable bladders connected to the pneumatic distribution system. The pneumatically inflatable bladders may be configured to inflate and press against one or more of the plurality of sensors and the at least one defibrillation electrode. The plurality of sensors may include pressure sensors integrated into the garment and configured to detect pressure from the garment on the patient. The controller may be configured to adjust a gas pressure in the pneumatically inflatable bladders, in response to a pressure sensor signal, via control of the gas flow regulator. At least a portion of the garment may include cooling tubes connected to the pneumatic distribution system. The cooling tubes may be configured to release compressed gas in a direction of the torso of the patient. The plurality of sensors may include moisture sensors configured to detect moisture on the skin of the patient. The controller may be configured to provide the compressed gas to the cooling tubes, in response to a moisture sensor signal, via control of the gas flow regulator. The at least one defibrillation electrode may include an inflatable cell connected to the pneumatic distribution system and an electrolyte gel sac in contact with the inflatable cell. The controller may be configured to increase a gas pressure in the inflatable cell in order to release electrolyte gel from the electrolyte gel sac via control of the gas flow regulator. The cardiac information of the patient may include one or more of electrocardiogram (ECG) information, pulse information, chest impedance information, and heart rate information. The plurality of sensors may include one or more of a blood pressure sensor, a motion sensor, a chest impedance sensor, a pulse oxygen level sensor, and a respiration rate sensor. At least a portion of the cuff may include at least one elastic strap. At least at least a portion of the cuff may include an adhesive surface. The cuff may be disposable. An external surface of the cuff may include a microporous fabric. The wearable medical treatment system may include a mobile power supply. The wearable medical treatment system may include a communications network interface coupled to the controller. The controller may be configured to transmit one or more of the cardiac information, an indication of delivery of the defibrillation shock, an indication of an implementation of the RIC protocol, or combinations thereof. The controller may be configured to receive the RIC protocol via the communications network interface. The controller may be configured to implement the RIC protocol prior to an expected cardiac event. The cardiac information may be predictive of the expected cardiac event.

A further example of a wearable medical treatment system for medical treatment of a patient according to the disclosure includes a plurality of sensors configured to be externally positioned on the patient, the plurality of sensors including at least one cardiac sensing electrode configured to provide one or more signals indicative of cardiac information of the patient, a cuff configured to contract about a limb of the patient, and a controller communicatively coupled to the plurality of sensors and the cuff. The controller is configured to receive the one or more signals indicative of the cardiac information of the patient, determine the cardiac information of the patient, and control contraction of the cuff based according to a remote ischemic conditioning (RIC) protocol and based at least in part on the cardiac information of the patient.

Implementation of such a system may include one or more of the following features. The RIC protocol may be determined based at least in part on the cardiac information of the patient. The controller may be configured to determine a cardiac event risk score associated with a potential adverse cardiac event of the patient based at least in part on the cardiac information of the patient. The cardiac event risk score may be indicative of a likelihood of a cardiac event in a time period after the determination of the cardiac event risk score. The cardiac event may include one or more of cardiac arrest and myocardial infarction. The controller may be configured to determine the RIC protocol based on the cardiac event risk score. The controller may be configured to implement the RIC protocol based on the cardiac event risk score. The RIC protocol may include a plurality of sequential treatment cycles and each cycle may include a cuff contraction phase during which the controller causes the cuff to contract about the limb of the patient to a set point pressure above a systolic pressure of the patient to occlude blood flow through the limb, an ischemic phase during which the controller causes the cuff to remain contracted about the limb of the patient at the set point pressure for an ischemic phase duration, a cuff release phase during which the controller causes the cuff to relax to allow the blood flow through the limb, and a reperfusion phase during which the controller causes the cuff to remain relaxed to allow the blood flow through the limb for a reperfusion phase duration. The wearable medical treatment system may include a gas pressure source, a gas flow regulator coupled to the gas pressure source and the controller, and a pneumatic distribution system coupled to the gas flow regulator. The cuff may include a pneumatically inflatable bladder removably connected to the pneumatic distribution system. The controller may be configured to adjust a gas pressure in the pneumatically inflatable bladder via control of the gas flow regulator. The cuff may include a band coupled to a mechanical tensioning mechanism. The wearable medical treatment system may include a communications network interface coupled to the controller. The controller may be configured to receive the RIC protocol via the communications network interface. The controller may be configured to transmit the cardiac information of the patient to one or more external computing devices via the communications network interface and to receive a cardiac event risk score via the communications network interface and the cardiac event risk score may be associated with a potential adverse cardiac event of the patient based at least in part on the cardiac information of the patient. The controller may be configured to implement the RIC protocol prior to an expected cardiac event and the cardiac information may be predictive of the expected cardiac event.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1 is a schematic diagram of a wearable medical treatment system.

FIG. 2 is an illustration of examples of cuff locations on a patient for RIC therapy.

FIG. 3 is a schematic diagram of the cuff in an unwrapped configuration.

FIG. 4 is a schematic diagram of system controller components.

FIGS. 5 and 6 are schematic diagrams of examples of cuff controller components.

FIG. 7 is a block diagram of a RIC cycle.

FIG. 8 is a block diagram of a method of providing cardiac and RIC therapy to a patient.

FIG. 9 is a flow chart of an example of an algorithm for determining cardiac event risk scores.

FIG. 10A is a bar graph representation of examples of cardiac event risk scores for multiple time periods.

FIG. 10B is a graphical representation of examples of historical information about the progression or change in cardiac event risk scores over time.

FIG. 11 is a flow chart of an example of a method for determining the cardiac event risk scores.

FIG. 12 is a flow chart of an example of an algorithm for determining cardiac event risk scores.

### DETAILED DESCRIPTION

Techniques are presented herein for providing cardiac therapy and reduced ischemic conditioning (RIC) therapy by a wearable medical treatment system. The system includes physiological sensors, including cardiac sensors that provide signals to a controller of the system. The controller processes these signals to determine cardiac information for the patient. Further, the controller may receive additional patient information such as medical history and patient activity information. The controller is configured to cause the medical treatment system to provide the cardiac therapy (e.g., pulsing and defibrillation) and/or the RIC therapy based on the cardiac information. The controller may also cause the system to provide therapy based on the additional patient information.

RIC is a therapeutic treatment that conditions the vasculature of a patient by temporarily and cyclically inducing ischemia. RIC induces ischemia (i.e., a localized shortage of oxygen-carrying blood supply) by occluding blood flow in the limb of the patient. The system may use information from the physiological sensors of the wearable system to tailor parameters of the RIC therapy to the needs of the patient. Further, the system may use the information from the physiological sensors to predict the possibility of a future medical event and, in response to the prediction, use RIC therapy to pre-condition the vasculature of the patient and thereby minimize, prevent or otherwise lessen the likelihood that the predicted medical event would occur. For example, the sensors may detect physiological parameters indicative of initial stages of a cardiac arrest or myocardial infarction. In response to this detection, the system described herein may provide the RIC therapy in an automated fashion in an attempt to prevent the actual cardiac arrest or myocardial infarction from happening. Thus the RIC described herein may be also be referred to as automated remote ischemic pre-conditioning (ARIPC).

Continuous monitoring of one or more physiological measurements or parameters may result in the detection of sudden changes indicative of an instability in the underlying state of the patient and/or a change in statistics of a series of data on the patient. A state change may occur days, hours, minutes, or seconds before a medical event, including sudden cardiac arrest, respiratory arrest, asystole, non-sudden death, or other medical events.

In response, the system may provide the RIC therapy at these initial stages and/or in response to indications of an elevated risk for these events. Such capability may enable the provision of the RIC therapy well before the system detects any symptoms of an occurring or impending event of the patient. Thus, rather than providing the RIC therapy as a response to a cardiac or other medical event, the described system provides the RIC therapy before the actual occurrence of the medical event. In some cases, early indicators of an impending cardiac event occur sporadically. Without the wearable medical system, the patient must rely on the unlikely scenario of these indicators coinciding with an examination by a physician.

As a further advantage, the integrated monitoring and RIC therapy system described herein allows for the application of the RIC therapy in an automated and timely manner. Providing the RIC therapy prior to the occurrence of the cardiac event in a timely and automated manner may significantly reduce the severity of the event, or may prevent the occurrence of the event altogether, and hence may lessen resulting damage to the physiological systems of the patient. For example, the system may implement the RIC therapy within seconds of detection of an elevated risk without the need for intervention by the patient and/or medical personnel. This may avoid delays which may otherwise be detrimental to the patient's health without the RIC therapy equipment. The wearable device also allows the patient to receive RIC therapy with minimal disruption of a daily routine. As a further benefit, the integrated system described herein allows provision of RIC therapy immediately prior to, immediately following, overlapping with and/or concurrently with provision of a defibrillation shock.

Other capabilities may be provided and not every implementation according to the disclosure must provide any, let alone all, of the capabilities discussed. Further, it may be possible for an effect noted above to be achieved by means other than that noted and a noted item/technique may not necessarily yield the noted effect.

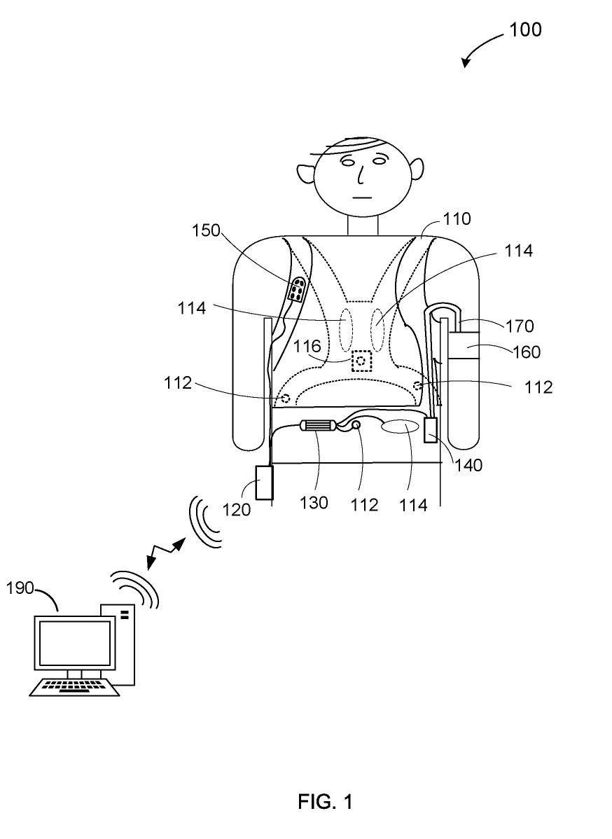

Referring to FIG. 1, a schematic diagram of a wearable medical treatment system is shown. The wearable system 100 is configured to be worn by a patient and to provide cardiac monitoring, cardiac therapy, and/or remote ischemic conditioning (RIC) therapy. In various implementations, the wearable system 100 may include functionality to administer treatments or interventions in addition to or as an alternative to pacing, defibrillation, and RIC. For example, the additional or alternative treatments or interventions may include automated drug delivery and/or automated administration of chest compressions based on communications with an automated chest compression delivery system (e.g., the ZOLL®AutoPulse®).

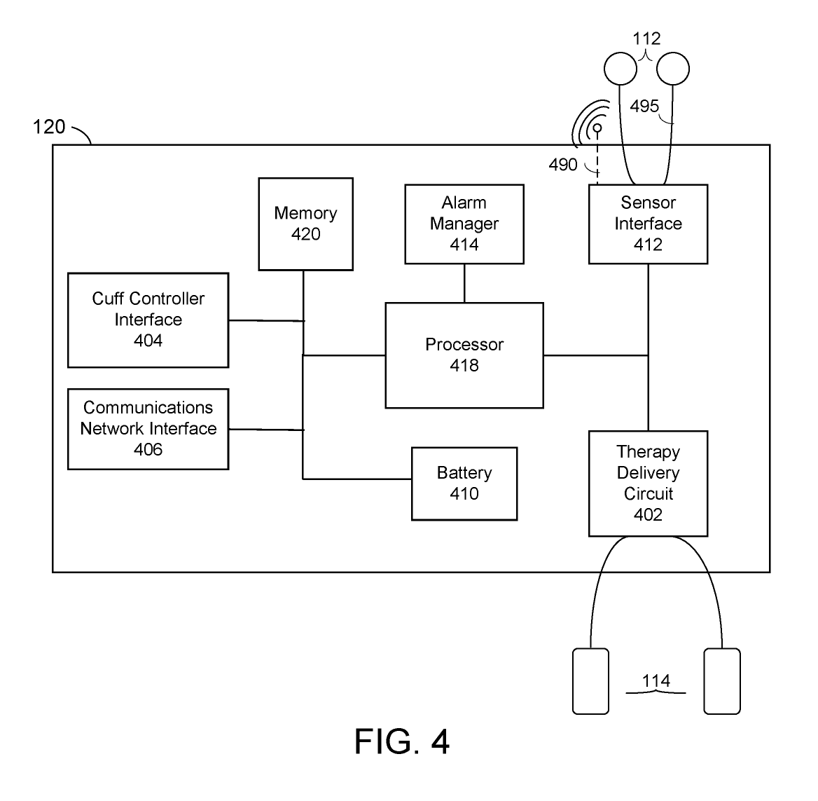

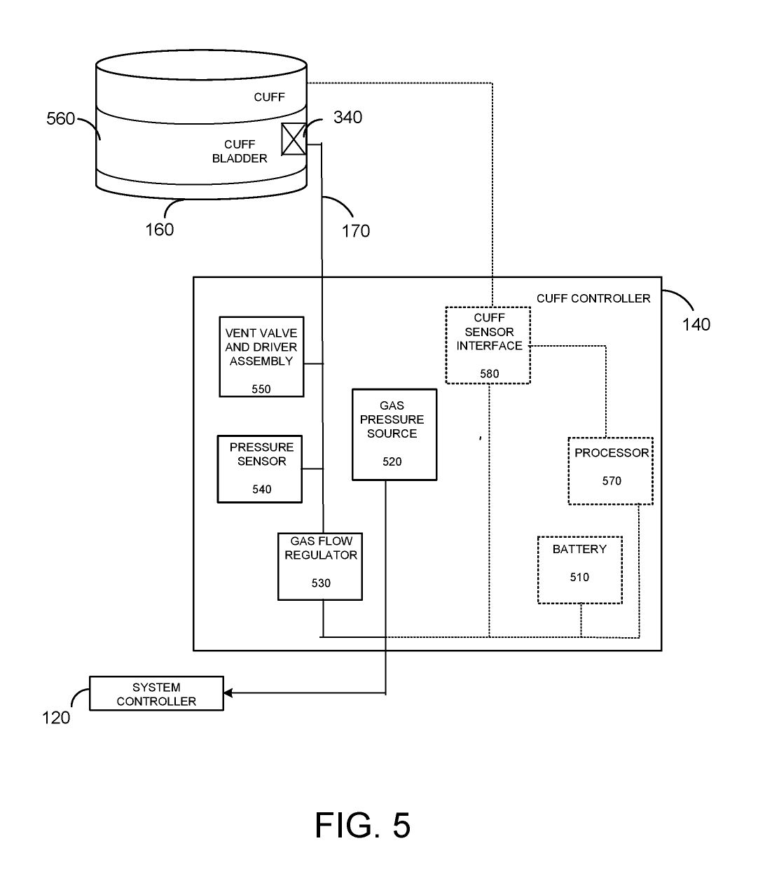

The wearable system includes a garment 110, one or more sensing electrodes 112, one or more therapy electrodes 114, a wearable medical device system controller 120, a connection pod 130, a user interface 150, a cuff controller 140, and a cuff 160. The system controller 120 is discussed in detail below in reference to FIG. 4 and the cuff controller is discussed in detail below in reference to FIGS. 5 and 6.

The garment 110 may be a vest, as shown for example in FIG. 1, a shirt, a jacket, or other garment configured to be worn on the torso of the patient. The vest may include a pair of shoulder straps and a belt that is worn about the torso of the patient. The locations of the components 112, 114, 120, 130, 150, 140, and 160 as shown in FIG. 1 with respect to the garment 110 and with respect to one another, are illustrative examples only and not limiting of the disclosure. Other arrangements of these components are consistent with the disclosure.

The components 112, 114, 120, 130, 150, 140, and 160 are coupled to the garment 110. One or more of these components may be coupled to the garment by being integrated into the garment 110. An integrated component is disposed in full or in part within a thickness of the garment 110 and/or constitute a portion of the garment 110. Alternatively or additionally, one or more of these components may be coupled to the garment by being attached or removably attached to a surface of the garment 110. A removably attached component is configured for removal and/or replacement without damage to the wearable system 100. Components may be removably attached to the garment 110 via, for example a hook and loop fastener, a snap, a button, an elastic strap, a buckled strap, a loop, a pouch, a holster, a belt, a clasp, a pin, a grommet, etc. or combinations thereof. In an implementation, one or more integrated components may couple to one or more surface components.

The one or more sensing electrodes 112 are configured to monitor and detect electrocardiogram (ECG) signals of the patient. For example, the one or more sensing electrodes 112 may be conductive and/or capacitive electrodes configured to measure changes in a patient's electrophysiology to measure the patient's ECG information. The sensing electrodes 112 may further measure the chest impedance of the patient. The sensing electrodes 112 may removably attach to the patient. For example, the sensing electrodes 112 may include an adhesive layer configured to stick to the skin of the patient. In an implementation, the sensing electrodes 112 may be disposable. The one or more sensing electrodes 112 may be dry-sensing capacitance electrodes and may include a front/back pair of ECG sensing electrodes and/or a side/side pair of ECG sensing electrodes. The wearable system 100 may include one or more additional physiological sensors 116 capable of monitoring the physiological condition or activity of the patient. For example, the additional physiological sensors 116 may provide signals indicative of blood pressure, heart rate, pulse oxygen level, respiration rate, heart sounds, lung sounds, respiration sounds, tidal CO₂, saturation of muscle oxygen (SMO₂), arterial oxygen saturation (SpO₂), cerebral blood flow, electroencephalogram (EEG) signals, brain oxygen level, tissue pH, tissue fluid levels, ultrasound images of the patient's heart, and/or patient movement.

The one or more therapy electrodes 114 are configured to deliver electrical current to the patient for defibrillation therapy and/or pacing therapy. The therapy electrodes 114 may be, for example, conductive metal electrodes such as stainless steel electrodes. In an implementation, one or more of the therapy electrodes 114 may include a gel deployment device. The gel deployment device is configured to deliver conductive gel to the therapy electrode 114 prior to delivery of the therapeutic shock. The therapy electrodes 114 may include one or more front electrodes and one or more back electrodes. The front electrodes are configured to attach to the front of the patient and the back electrodes are configured to attach to the back of the patient. In an implementation, the therapy electrodes 114 may be a therapy electrode assembly that includes a pair of electrically coupled therapy electrodes 114. The pair of therapy electrodes 114 may enable provision of a biphasic shock to the patient. For example, a first electrode of the pair of therapy electrodes 114 may deliver a first phase of the biphasic shock with the second therapy electrode of the pair of therapy electrodes 114 acting as a return. Subsequently, the second therapy electrode may deliver a second phase of the biphasic shock with the first therapy electrode acting as the return.

In an implementation, a user and/or a provider of the wearable system 100 may disable the therapy electrodes 114. For example, the user and/or the provider may remove the therapy electrodes 114 from the garment 110 and/or may change a hardware and/or software setting in the system controller 120 to disable operation of the therapy electrodes 114. The garment 110 may thus be a component of a wearable system that further includes one or more sensing electrodes 112, a wearable medical device system controller 120, a connection pod 130, a user interface 150, a cuff controller 140, and a cuff 160. The wearable system 100 may be smaller, lighter and more comfortable for the patient without the therapy electrodes 114 and associated shock delivery components (e.g., the system controller 120 may not include the therapy delivery circuit 402 as described below with regard to FIG. 4). For example, the patient may wear the wearable system 100 without the defibrillator portion when they are in the shower or swimming. A wearable system 100 that does not include the defibrillator portion (e.g., does not include, or otherwise deactivates, the therapy delivery circuit 402 and/or the electrodes 114) may be referred to as a cardiac monitoring device or system.

Although shown as separate components in FIG. 1, in an implementation, a single integrated electrode may include a therapy electrode 114 and a sensing electrode 112. The monitoring and therapy provided by electrodes 112 and 114 may treat cardiac conditions including, but not limited to, atrial fibrillation, bradycardia, tachycardia, atrio-ventricular block, Lown-Ganong-Levine syndrome, atrial flutter, sino-atrial node dysfunction, cerebral ischemia, syncope, atrial pause, and/or heart palpitations.

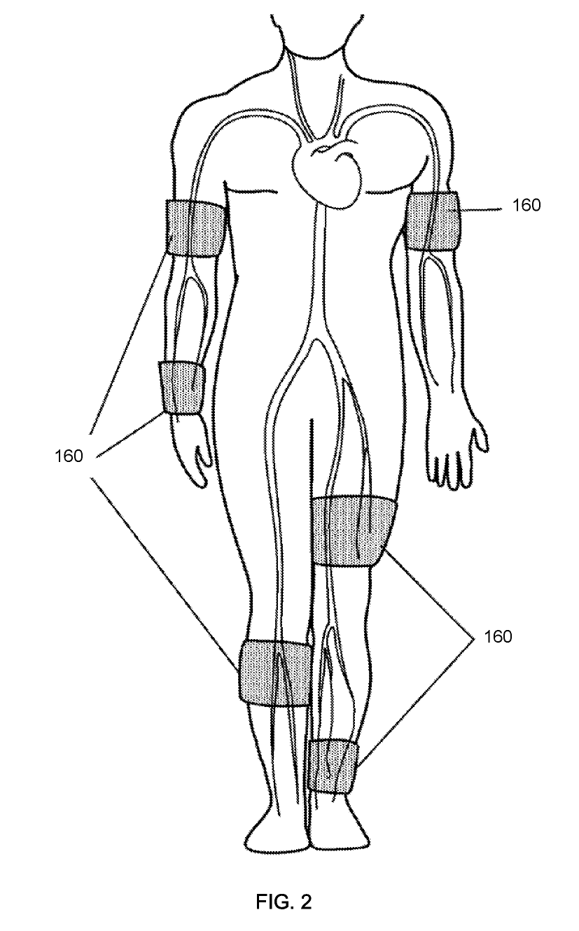

The cuff 160 is shown in FIG. 1 disposed on the upper arm of the patient as an example only. The cuff 160 is configured to provide RIC therapy under control of the cuff controller 140 and the system controller 120. In various implementations, the wearable system 100 may include one or more cuffs disposed on one or more of the upper arm, the lower arm, the thigh, the calf, the ankle, and portions thereof. The placement of the cuff may provide occlusion during a RIC treatment protocol of, for example, one or more of the brachial artery, the radial artery, the ulnar artery, the femoral artery, the deep femoral arteries, and the tibial arteries. Examples of cuff locations on the patient for RIC therapy are shown in FIG. 2. The cuffs in FIG. 2 are shown on the limbs of the patient. In the example of FIG. 2, the cuffs are located on the upper arm (e.g., surrounding the brachial artery), on the forearm (e.g., surrounding the radial artery and/or the ulnar artery), on the thigh (e.g., surrounding the femoral artery and/or the deep femoral arteries), and on the calf (e.g., surrounding the tibial arteries). The calf location examples include the upper calf (e.g., just below the knee), and the lower calf (e.g., proximate to the ankle).

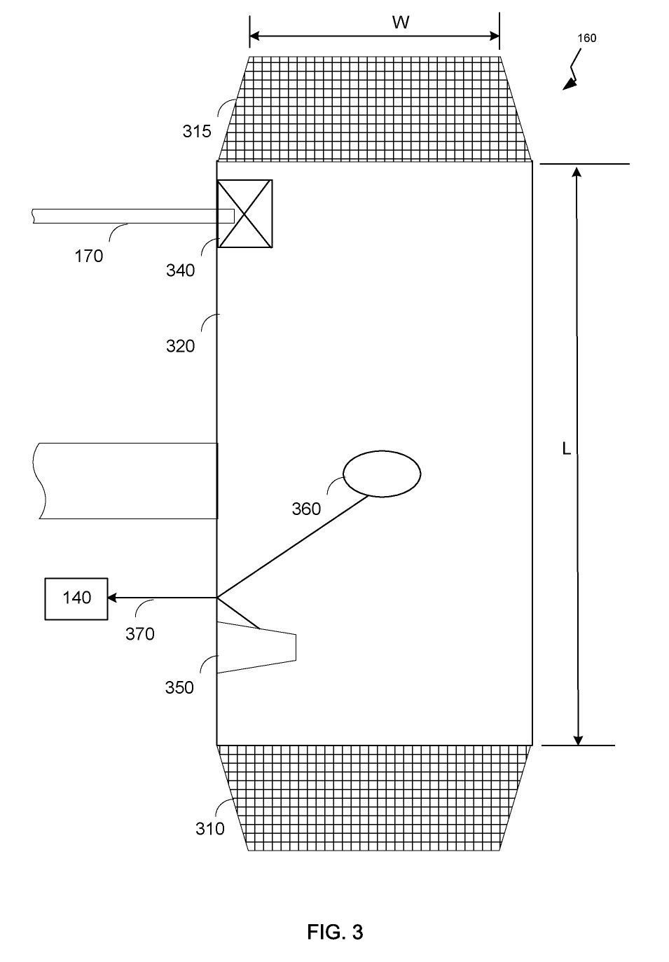

Referring to FIG. 3, in accordance with various embodiments of the present disclosure, a schematic diagram of the cuff in an unwrapped configuration is shown. The cuff 160 is configured to be positioned about a limb of the patient and to contract about the limb when actuated. In order to use the cuff 160, the patient and/or a caregiver wraps the cuff 160 around the limb and fastens the cuff 160 so that it fits snuggly around the limb. A cuff controller 140 (e.g., as described in more detail below in reference to FIG. 5) inflates the cuff such that the limb is constricted to the point of occluding blood flow through the limb.

The cuff 160 may include one or more attachment sections 310 and 315 and an inflatable section 320. The attachment sections 310 and 315 are non-inflatable and are configured to secure the cuff 160 around the limb of the patient. The attachment sections 310 and 315 may include for example elastic straps, hook and loop fasteners, buckles, etc. In an implementation, the cuff 160 may include an adhesive surface to couple the cuff 160 to the patient's limb. In an implementation, the cuff 160 may removably couple to the garment 110 via one or more attachment straps 330 that includes example a hook and loop fastener, a snap, a button, an elastic strap, a buckled strap, a clasp, etc. or combinations thereof. In an implementation, the cuff 160 may be modular and removable from the garment 110. For example, the patient may remove the cuff 160 for cleaning or for periods of time when the therapy provided by the cuff 160 is unneeded. As another example, the cuff 160 may be disposable and the patient may remove the cuff 160 to dispose of and replace the cuff 160.

The inflatable section 320 includes a cuff bladder (not shown) disposed inside of a cuff sleeve that receives a fluid, such as air or other gas, to cause the cuff expand and retract about the patient's limb. The bladder is constructed of an air impermeable material, such as flexible plastic or rubber. Although the illustrated implementation includes a single bladder positioned within a cuff, it is to be appreciated that other implementations are also possible. By way of example, according to some implementations, the fabric sleeve may itself be air impermeable, such that no separate bladder is required. In other implementations, multiple, separate inflatable bladders may be incorporated into a common sleeve, as aspects of the present invention are not limited in this respect. In an implementation, the cuff sleeve may itself be air impermeable, such that no separate bladder is required. Alternatively, the cuff sleeve may include a microporous fabric and, therefore, require the separate bladder.

The cuff bladder may fill all or a portion of the inflatable section 320. In various implementations, the cuff 160 may include two or more bladders. The two or more bladders may have different shapes and/or sizes and may be configured with combinations of shapes and/or sizes. Each bladder may be generally inflated and deflated via its own dedicated air handling circuit. This arrangement allows for both independent or simultaneous inflation and deflation of the two or more bladders. In an implementation, the bladder may fill substantially all of the inflatable section 320 of the cuff 160 and is configured to compress the limb of the patient around approximately the full circumference of the limb. In an implementation, the bladder is confined to a portion of the inflatable section 320 of the cuff 160 and is configured to occlude of arterial flow in a particular portion of the limb. For example, the cuff 160 may compress the inner surface of the arm of the patient (i.e., the cuff bladder is configured to be proximate to the surface of the arm that approximately faces the torso) more than the outer surface of the arm.

Dimensions of the cuff 160 (e.g., length L and width W) may generally follow established standards established for blood pressure measurement cuffs. In various implementations, the dimensions of the cuff 160 may be larger or smaller than standard dimensions for the blood pressure cuff. Standard dimensions for the blood pressure cuff may apply to arm cuffs. The RIC treatment cuff designed for use on the thigh, calf, and/or ankle may require different dimensions in order to fit securely around the thigh, calf, and/or ankle. As another example, the cuff 160 may be configured to cover substantially the entire upper arm between the shoulder and the elbow, substantially the entire lower arm between the elbow and the wrist, substantially the entire thigh, or substantially the entire calf. In an implementation, the size of the cuff 160 may be adjustable to accommodate a range limb girths. In other example configurations, the cuff 160 may be long enough to accommodate a limb circumference of three to four feet.

The cuff 160 includes a connection port 340 that is present at one end of the bladder to allow air to enter the bladder during inflation and to exit the bladder during deflation. The port may include engagement features to facilitate a connection to the cuff controller 140, such as by an air hose. These features may include threads, clips, and the like. The connection port 340 removably connects the cuff bladder to a pneumatic distribution system. The connection port 340 may include a valve configured to disable/enable gas flow to the bladder. For simplicity, in FIGS. 1 and 3, a single gas flow tube 170 is shown. However, the wearable system 100 may include multiple gas flow tubes that form a pneumatic distribution system attached to and/or integrated into the garment 110. In an implementation, the cuff 160 may include a side port 350 configured to removably attach to a manual or automated blood pressure measurement device such as a patient monitor. In an implementation, the cuff 160 may include one or more blood pressure sensors 360 configured to monitor the limb for the onset of Korotkoff sounds or vibrations.

In an implementation, the cuff 160 may include an electrical coupling 370 to the cuff controller 140. The cuff controller 140 may receive signals indicative of blood pressure measurements as determined by the blood pressure measurement device and/or signals from the one or more blood pressure sensors 360 via the electrical coupling 370. The cuff controller 140 may send these signals to the system controller 120 as feedback.

Referring again to FIG. 1, the system controller 120 may control operation of the sensing electrodes 112, the therapy electrodes 114, and the cuff 160. The system controller 120 may control these operations via wired and/or wireless connections to the various controlled components. In an implementation, the system controller 120 may transmit and/or receive electrical signals to and/or from the various controlled components via the connection pod 130. The controller 120 may communicate with one or more external computing devices 190 via a wired and/or wireless communicative connection. The one or more external computing devices 190 may include, for example, a server, a personal computer, a laptop computer, a mobile device, a hand-held device, a wireless device, a tablet, a medical device, a defibrillator, a patient monitor, a wearable device (e.g., a wrist-worn device, a head-worn device, etc.), or combinations thereof. The server may be a cloud server or a server associated with a central facility such as a hospital, a physician's office, a medical records office, an emergency services office, an emergency services vehicle, a dispatch center, etc.

The connection pod 130 electrically couples the plurality of sensing electrodes 112 and the therapy electrodes 114 to the system controller 120, and may include various electronic circuitry. For example, the connection pod 130 may include a signal processor and other signal acquisition circuitry configured to amplify, filter, and/or digitize signals from the sensors 112 and/or other components prior to transmitting these signals to the system controller 120. As a further example, the connection pod 130 may include a motion sensor (e.g., an accelerometer and/or a gyroscope) configured to monitor patient activity. The sensing electrodes 112 and/or the additional sensors may provide the system controller 120 with physiological signals at periodic or aperiodic time intervals and times. These time intervals and/or times may be pre-determined and/or user input or other events may trigger monitoring.

Referring to FIG. 4, a block diagram of system controller components is shown. The system controller 120 includes a processor 418, a memory 420, a sensor interface 412, a therapy delivery circuit 402, a communications network interface 406, a battery 410, and a cuff controller interface 404. In general, the system controller 120 is configured to monitor the patient's medical condition, to perform medical data logging, storage, and communication, and to provide medical treatment to the patient in response to a detected and/or predicted medical event or conditions.

The components 402, 404, 406, 412, 414, and 420 are communicatively coupled to the processor 418 for bi-directional communication. Although shown as separate entities in FIG. 4, the components 402, 404, 406, 412, 414 may be combined into one or more discrete components and/or may be part of the processor 418. The processor 418 and the memory 420 may include and/or be coupled to associated circuitry in order to perform the functions described herein.

The processor 418 is a physical processor (i.e., an integrated circuit configured to execute operations off the system controller 120 specified by software and/or firmware). The processor 418 may be an intelligent hardware device, e.g., a central processing unit (CPU), one or more microprocessors, a controller or microcontroller, an application specific integrated circuit (ASIC), a general-purpose processor, a digital signal processor (DSP), or other programmable logic device, a state machine, discrete gate or transistor logic, discrete hardware components, or any combination thereof designed to perform the functions described herein and operable to carry out instructions on the system controller 120. The processor 418 may utilize various architectures including but not limited to a complex instruction set computer (CISC) processor, a reduced instruction set computer (RISC) processor, or a minimal instruction set computer (MISC). In various implementations, the processor 418 may be a single-threaded or a multi-threaded processor. The processor 418 may be implemented as a combination of computing devices (e.g., a combination of DSP and a microprocessor, a plurality of microprocessors, one or more microprocessors in conjunction with a DSP core, or any other such configuration). The processor 418 is configured to execute processor-readable, processor-executable software code containing one or more instructions or code for controlling the processor 418 to perform the functions as described herein. The processor 418 may one or more processors (or one or more processor cores) that each are configured to perform a series of instructions that result in manipulated data and/or control the operation of the other components of the system controller 120. The processor 418 may include multiple separate physical entities that may be distributed in the system controller 120. In some example cases, the processor 418 can proceed through a sequence of logical transitions in which various internal register states and/or other bit cell states internal or external to the processor 418 may be set to logic high or logic low. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications. In some implementations, the processor is configured to make specific logic-based determinations based on input data received and, further, to provide one or more outputs that may control or otherwise inform subsequent processing by the processor 418 and/or other processors or circuitry with which processor 418 is communicatively coupled. Thus, the processor 418 can react to specific input stimulus in a specific way and generates a corresponding output based on that input stimulus.

In an implementation, the processor 418 is configured to determine a location of the patient. For example, the processor 418 may include global positioning system (GPS) and/or indoor positioning system capabilities. The processor 418 may also be configured to receive location input from the patient via the user interface 150.

The processor 418 is operably coupled to the memory 420. The memory 420 refers generally to a computer storage medium, including but not limited to RAM, ROM, FLASH, fuse devices, and portable storage media, such as Universal Serial Bus (USB) flash drives, etc. The USB flash drives can store operating systems and other applications. The USB flash drives can include input/output components, such as a wireless transmitter and/or USB connector that can be inserted into a USB port of another controller and/or a computing device. The memory 420 may be long term or short term and is not to be limited to any particular type of memory or number of memories, or type of media upon which memory is stored. The memory 420 includes a non-transitory processor-readable storage medium (or media) that stores the processor-readable, processor-executable software code.

The sensor interface 412 is configured to couple to one or more sensors associated with the wearable system 100. The sensors may be coupled to the wearable medical device system controller 120 via a wired connection (e.g., connections 495, 496) or wireless connection (e.g., connection 490). The sensor interface 412 is configured to receive information descriptive of physiological parameters of the patient from the one or more sensors. The sensors can include the one or more sensing electrodes 112 and/or the additional physiological sensors 116 (e.g., as described above with regard to FIG. 1). The sensor interface 412 can provide received information to the processor 418 for analysis of the received information.

The therapy delivery circuit 402 is coupled to the one or more therapy electrodes 114. For example, the therapy delivery circuit 402 can include, or be operably connected to, circuitry components that are configured to generate and provide pacing and/or defibrillation pulses. For example, the therapy delivery circuit 402 may include a one or more capacitors configured to store electrical energy for a pacing pulse or a defibrillating pulse. The therapy delivery circuit 402 may be configured to connect the capacitors in parallel for charging the capacitors and to switch the connection to a series connection for discharging the capacitors. Discharging the capacitors provides the therapeutic pulse. For example, the circuit 402 may include four capacitors of approximately 650 microfarads. The capacitors may have between 350 to 500 volt surge rating. The battery 410 may charge the capacitors in approximately 15 to 30 seconds. The circuit components may further include, for example but not limited to, resistors, additional capacitors, relays and/or switches, electrical bridges such as an H-bridge (e.g., including a plurality of insulated gate bipolar transistors or IGBTs), voltage measuring components, and/or current measuring components.

Pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation pulses can be used to treat ventricular tachycardia and/or ventricular fibrillation. Each defibrillation pulse may deliver between 60 to 180 joules of energy. In some implementations, the defibrillating pulse may be a biphasic truncated exponential waveform. The term “biphasic” indicates that the pulse signal alternates between a positive charge direction and a negative charge direction where the direction of electrical current flow through the body in a first phase is opposite the direction of electrical current flow through the body in a second phase. The biphasic waveform may provide an effective defibrillation pulse at a lower energy as compared to a monophasic waveform. In an implementation, the topology of the therapy delivery circuit 402 is configured to automatically adjust an amplitude and a width of the two phases of the energy waveform to deliver a substantially constant amount of energy (e.g., 150 joules) regardless of the patient's body impedance.

The communications network interface 406 may transmit and/or receive information from and/or to one or more computing devices external to the system controller 120. The communications network interface 406 may transmit and/or receive the information via a wired and/or a wireless communicative connection to the one or more external computing devices 190. The received and/or transmitted information may include information determined and/or received by the system controller 120 and/or information stored in the memory 420. This information may include, for example, but not limited to, monitoring information, treatment information, patient information, location information, cardiac information (e.g., cardiac information determined from one or more signals from the sensors 112 and/or 116), in indication of a delivery of a defibrillation shock, and in indication of an implementation of a RIC protocol. The communications network interface 406 may access a communications network, including, for example, but not limited to, a local area network, a cellular network, and/or a computer network (e.g., an Internet Protocol network). The communications network interface 406 may provide Wi-Fi, Bluetooth®, satellite, and/or cellular network communications capabilities.

In an implementation, the one or more external computing devices 190 may include a computing device associated with a manned monitoring center. Cardiac-trained reviewers and/or caregivers associated with the center may interpret the data. Further, the center may provide feedback to the patient and/or a designated caregiver via detailed periodic or event-triggered reports. In an implementation, the patient may report a symptom of the patient via the user interface 150. For example, the symptom may be a skipped beat, shortness of breath, light headedness, racing heart rate, fatigue, fainting, chest discomfort, weakness, dizziness, and/or giddiness. The system controller 120 may record monitored physiological signals and/or the recorded symptoms. In an implementation, the controller may record and/or transmit to the remote server predetermined physiologic parameters of the patient (e.g., ECG information) for a predetermined amount of time before and/or after the reported symptom (e.g., 1-30 minutes before and/or 1-30 minutes after a reported symptom).

The battery 410 is a mobile power supply configured to provide power to one or more components integrated in and/or connected to the system controller 120. The battery 410 may include a rechargeable multi-cell battery pack. The type of battery may include, for example, but not limited to lithium ion, nickel-cadmium, or nickel-metal hydride batteries. In an implementation, the battery 410 may include three or more 2200 mAh lithium ion cells. The battery 410 may provide its power output in a range of between 20 mA to 1000 mA (e.g., 40 mA) output and may support 24-100 hours, or more depending on usage, of runtime between charges.

The alarm manager 414 is configured to manage alarm profiles and notify one or more intended recipients of events specified within the alarm profiles as being of interest to the intended recipients. These intended recipients may include external entities such as users (e.g., patients, physicians, and monitoring personnel) as well as computer systems (e.g., monitoring systems, emergency response systems). The alarm manager 414 may be implemented using hardware or a combination of hardware and software. For example, the alarm manager 414 may be implemented as a software component that is stored within the memory 420 and executed by the processor 418. In this example, the instructions included in the alarm manager 414 can cause the processor 418 to configure alarm profiles and notify intended recipients using the alarm profiles. As another example, the alarm manager 414 may be an application-specific integrated circuit (ASIC) that is coupled to the processor 418 and configured to manage alarm profiles and notify intended recipients using alarms specified within the alarm profiles. Thus, examples of alarm manager 414 are not limited to a particular hardware or software implementation.

The user interface 150 is communicatively and/or electrically coupled to the system controller 120. The user interface 150 is configured to attach to the patient's clothing or body or to the garment 110. For example, the user interface 150 may include a clip (not shown) or other attachment mechanism. Alternatively, the patient or a caregiver may hold the user interface 150. The user interface 150 may communicate wirelessly with the system controller 120, for example, using a Bluetooth®, Wireless Universal Serial Bus, ZigBee®, Wireless Ethernet, cellular or other type of communication interface. The user interface 150 may include one or more buttons or a touch screen by which the patient or a bystander may communicate with the system controller 120, and a speaker and/or a display by which the system controller 120 may communicate with the patient or the bystander. For example, if the system controller 120 determines that the patient is experiencing a cardiac arrhythmia, the system controller 120 may issue an audible alarm via a loudspeaker (not shown) on the system controller 120 and/or the user interface 150 alerting the patient and any bystanders to the patient's medical condition. The system controller 120 may also instruct the patient to press and hold one or more buttons on the system controller 120 or on the user interface 150 to indicate that the patient is conscious, thereby instructing the system controller 120 to withhold the delivery of one or more therapeutic defibrillating shocks. If the patient does not respond, the device may presume that the patient is unconscious, and proceed with a defibrillation treatment sequence, culminating in the delivery of one or more defibrillating shocks to the body of the patient. In an implementation, functionality of the user interface 150 may be integrated into the system controller 120. In an implementation, input to the user interface 150 may provide manual control of inflation and/or deflation of the cuff 160. In an implementation, the user interface 150 may include an emergency override that causes the system controller 120 to release the cuff pressure. For example, the user interface 150 may include a button or other mechanical switch, a touch screen icon or button, a voice activated control, etc. that provides an emergency venting signal to the system controller 120 and/or the cuff controller 140 to vent the cuff 160.

Referring to FIG. 5, a block diagram of an example of cuff controller components is shown. The system controller 120 may include a cuff controller interface 404. The cuff controller interface 404 is configured to provide control signals from the system controller 120 to the cuff controller 140. In an implementation, the cuff controller interface 404 may receive feedback signals from the cuff controller 140. The control signals may control the components of the cuff controller 140 to start and stop flow and regulate inflation and deflation parameters for the cuff 160. The inflation and deflation parameters may include one or more of an inflation rate, a deflation rate, an inflation pressure, and timing associated with inflation, pressure maintenance, and venting. The feedback signals may include pressure signals from the pressure sensor 540, blood pressure signals from blood pressure sensors associated with the cuff 160 and/or other signals indicative of the functions of the components of the cuff controller 140 and the cuff 160. The cuff controller interface 404 may be communicatively and/or electrically coupled to the cuff controller 140. The communicative coupling may be a wired and/or a wireless connection. The cuff controller 140 includes a gas pressure source 520, a gas flow regulator 530, a pressure sensor 540, and a vent valve 550.

The gas pressure source 520 is configured to provide gas to the cuff bladder 560 to inflate the cuff bladder 560. In the illustrated embodiment, the gas pressure source 520 is a device configured to deliver a fluid, such as air or gas, to the gas flow tube 170. For simplicity, and similarly to FIGS. 1 and 3, a single gas flow tube 170 is shown. However, the wearable system 100 may include multiple gas flow tubes that form a pneumatic distribution system attached to and/or integrated into the garment 110. For example, the gas pressure source 520 may be, for example a gas pump (e.g., a piston compressor pump, a diaphragm pump, a centrifugal pump, a scroll compressor, an air turbine driven by an electrical motor, etc.) or a cartridge containing compressed gas. The gas pump may be configured to provide air flow at a rate of between 0.1 to 20 cubic feet per minute, with a head pressure of up to 50 psi, according to some implementations. However, other flow rates and/or pressures are possible, as aspects of the disclosure are not limited in this respect. As another example, the gas pressure source 520 may be a cartridge containing compressed gas. As a further example, the gas pressure source 520 may be a sealed container including components for a gas-producing chemical reaction. In an implementation, the gas pressure source 520 may be external to the cuff controller 140. For example, the cuff controller 140 may connect to a compressed gas cartridge, a gas supply from another medical device, a gas supply from a patient monitor, a wall source of compressed gas, or a pump controlled manually based on instructions provided to the pump user from the system controller 120. In an implementation, instead of the gas pressure source, the cuff controller 140 may include a pressurized liquid source and regulator along with a bleed valve for inflation and deflation of the bladder.

The gas flow regulator 530 is configured to control the rate of inflation. In an implementation, the gas pressure source 520 and gas flow regulator 530 may be configured and/or controlled to provide a slower rate of cuff inflation as compared with traditional blood pressure measuring devices. Traditional blood pressure measuring devices are typically designed to complete the act of inflating the blood pressure cuff as quickly as possible (e.g., 3-10 seconds) so as to rapidly provide the reading to the medical practitioner and avoid measurement errors caused by motion of the patient. Such a rate of inflation may not be needed for RIC. Therefore, the gas pressure source 520 and gas flow regulator 530 may be configured and/or controlled to complete the inflation of the cuff over a longer period of time (e.g., 20-60 seconds). Slower rates of inflation (e.g., less than about 0.1 cubic foot per minute) may enable a reduction in the weight and/or size of the gas pressure source 520. Variable rates of inflation may improve the accuracy of the pressure measurement (e.g., by the pressure sensor 540) and/or may improve the control of timing of changes in the cuff pressure with regard to physiological events for the patient.

The pressure sensor 540 is configured to measure the cuff bladder pressure and provide signals indicative of bladder pressure to the cuff controller 204. The cuff controller 140 may control the gas flow regulator 530 based on the received signals indicative of bladder pressure to inflate and deflate the cuff bladder 560. Cuff pressure is often directly indicative of the pressure that exists within a blood vessel of the limb beneath the cuff. The system controller 120 may be programmed to target a particular cuff pressure that is to be maintained during an ischemic duration of a RIC cycle, as is discussed in further detail below. Although shown as a component of the cuff controller 140, the pressure sensor 540 may be positioned anywhere within the pressurized space of the cuff or in the pneumatic distribution system. In addition to the pressure sensor 540, one or more additional pressure sensors may also be positioned on an inner surface of the cuff 160 to directly measure the pressure between the cuff and an outer surface of the patient's limb. In use, the cuff may be oriented such that the pressure sensor is positioned directly above the patient's artery, so as to provide a more direct measurement of pressure at a blood vessel of interest.

The vent valve 550 is configured to vent the cuff bladder 560 to an atmospheric or ambient level. In an implementation, the vent valve 550 may be configured to provide a controllable rate of venting. By way of example, a slow release valve may be incorporated into a pneumatic cuff to provide for a continual, slow release of pressurized air from the cuff. The continual slow release mechanism may provide for the safe release of the patient's limb, even in the face of power failures or other events that may prevent redundant safety features from operating properly. The release valve, as shown, may be a solenoid that moves rapidly between fully closed and fully open positions to rapidly release air from the cuff and, in turn, to rapidly release the cuff from the patient. According to some implementations, the same release valve or another release valve may also be actuated to open slowly, such as to adjust the pressure of the cuff or to allow a more controlled release of pressure such as may be required when the patient's blood pressure is measured.

Implementations of the system may include safety features to allow rapid release of the cuff from the patient's limb. Moreover, some of these implementations may be readily activated by the patient, such as when the patient feels discomfort. In one implementation, the safety release includes a large button positioned on or near the cuff. In this regard, the safety release is within reach of the patient. In other implementations, the safety release may comprise a separate actuator, such as one that may be held in the free hand of the patient. Activating the safety release may cause the release valve of a pneumatic cuff to open, thereby allowing rapid removal of air from the cuff.

In an implementation, the cuff controller 140 may include a processor 570. The processor 570 may supplement the system controller 120. For example, the processor 570 may monitor the performance of the system controller 120 and/or the cuff controller 140. Further, the processor 570 may assure proper functioning of the vent valve 550. In an implementation, the processor 570 is programmed to open the vent valve 550 to ensure patient's safety in the event of a malfunction of the system controller 120. In an implementation, the processor 570 may control the cuff controller 140 to implement RIC therapy independently from the system controller 120 (e.g., without receiving control signals from the system controller 120). In such an implementation, the processor 570 may be communicatively coupled (e.g., via a wired or wireless connection) to the system controller 120 and may provide RIC therapy implementation information to the system controller 120.

In an implementation, the cuff controller 140 may include a cuff sensor interface 580. The cuff sensor interface 580 is configured to receive measurements from the cuff and signals from cuff sensors including, for example, blood pressure measurements and/or blood pressure sensor signals. The cuff sensor interface 580 may receive signals indicative of blood pressure measurements as determined by a blood pressure measurement device connected to the cuff 160 and/or signals from the one or more blood pressure sensors 360. The signals from the one or more blood pressure sensors 360 may be indicative of Korotkoff sounds or vibrations. The cuff controller 140 may send these signals to the system controller 120 as feedback. Alternatively or additionally, the cuff sensor interface 580 may provide these signals to the processor 570.

Other optional features may include a manual user input capability to enter a user-selected limit of cuff inflation pressure. If the device cannot detect the blood pressure of the patient, an override may be included allowing the user to select the upper level of cuff inflation. The function of the device may be limited in this case as no automatic monitoring of hemodynamics would be conducted. At the same time, the primary purpose of the device, namely providing ischemic preconditioning will still be enabled.

In an implementation, the battery 410 of the system controller 120 may provide power to the cuff controller 140. Alternatively or additionally, the cuff controller 140 may include a battery 510. In an implementation, the battery 510 may solely provide power to the cuff controller 140. In a further implementation, the battery 510 may supplement power provided by the system controller 120 and/or serve as a back-up power source to the system controller 120. The battery 510 may include one or more lithium ion batteries and/or other rechargeable and/or replaceable batteries.

In an implementation, in lieu of the pneumatic controller, the wearable system 100 may include a mechanical control system configured to tighten and loosen a number of times around the patient's limb in response to control signals from the system controller 120. The mechanical control system may include a ratcheting or other tensioning mechanism. For example, the band may include a tensioning mechanism configured to move one end of the band relative to other portions of the band so as to place the band in tension. For example, such a mechanism may include opposed rollers within a housing. The housing may include a slot for receiving a free end of the band and a fixation point for fixed attachment to the opposite end of the band. The free end of the band is passed into the slot and between the rollers. The rollers may be mechanically actuated to rotate relative to one another, such as by an electric motor, to pull the free end through the housing and thus tighten the band around the patient's limb.

Referring to FIG. 6, a schematic diagram of an example of cuff controller components is shown. In an implementation, the cuff controller 140 (e.g., as described above with reference to FIG. 5) may include additional components that allow the cuff controller 140 to function as a pneumatics controller 640 for the cuff 160 and the garment 110. Further, the garment 110 may include one or more of one or more pneumatically inflatable bladders 680 and one or more cooling tubes 690. Thus the pneumatics controller 640 may control the cuff 160, the bladder(s) 680, and/or the tube(s) 690. Although shown as a single pneumatics controller 640 for simplicity, the described functions and/or hardware associated with the pneumatics controller 640 may be distributed across and/or associated with one or more controllers. In an implementation, the system controller 120 may include one or more components shown in FIG. 6 as being part of the pneumatics controller 640 and/or may provide one or more functions described below as being functions of the pneumatics controller 640.

The capabilities and functions of the pneumatics controller 640 include those capabilities and functions described above with regard to the cuff controller 140. Further, the cuff controller interface 404 (e.g., shown in FIG. 4) may be configured to provide control signals from the system controller 120 to the pneumatics controller 640 and to receive feedback signals from the pneumatics controller 640 (e.g., as described above with regard to the cuff controller 140).

The garment 110 may include one or more pneumatically inflatable bladders 680 integrated into and/or attached to the garment 110. These bladders 680 may be disposed at various locations on the garment. The one or more garment bladders 680 are coupled to one or more gas flow tubes 670. In an implementation, multiple garment bladders 680 are coupled to one gas flow tube 670. Alternatively, each garment bladder 680 is coupled to a dedicated gas flow tube 670. Each gas flow tube 670 connects to a gas flow regulator 632, a pressure sensor 642, and a vent valve 652. The components 632, 642, and 652 function substantially as described above in reference to FIG. 5 with regard to the components 530, 540, and 550. In an implementation, the gas flow regulator 632 is coupled to multiple gas flow tubes and multiple pressure sensors 642 and/or vent valves 652 in a configuration that allows multiple bladders to be inflated and deflated at different times with respect to one another. Alternatively or additionally, the configuration may allow individual control of the pressure of one or more of the multiple bladders.

The garment bladders 680 may provide the garment 110 with a variety of capabilities. As an example, one or more of the garment bladders 680 may be configured to press against one or more of the sensing electrodes 112, the additional physiological sensors 116, and/or the therapy electrodes 114 when inflated. In this manner, the inflated garment bladder 680 may improve the contact between the sensors or electrodes and the skin of the patient. As another example, the garment 110 may include integrated and/or attached pressure sensors configured to measure the pressure from the garment 110 on the patient. In general, a snug fit of the garment 110 is desirable for proper functioning of the monitoring and therapy systems. However, the patient may gain or lose weight and/or the garment 110 may stretch, or possibly shrink during use. In these cases, the garment 110 may be too loose or too tight on the patient. The garment bladders 680 may enable automated size adjustment of the garment 110. For example, the garment bladders 680 may fully or partially inflate or deflate to adjust the garment size in response to an indication from the pressure sensors that the garment is too tight or too loose. These pressure sensors may be coupled to the sensor interface 666 of the pneumatics controller 640.

In a further example, one or more of the garment bladders 680 may be an inflatable cell coupled to one or more of the therapy electrodes 114. The one or more of the therapy electrodes 114 may further include an electrolyte gel sac in contact with the inflatable cell. In such an example, the system controller 120 is configured to control the cuff controller 140 to increase a gas pressure in the inflatable cell in order to release electrolyte gel from the electrolyte gel sac.

In an implementation, the garment 110 may include one or more cooling tubes 690. The cooling tubes 690 are configured to release compressed gas in a direction of the torso of the patient (e.g., towards the skin or clothing of the patient) in order to provide cooling. The garment 110 may include temperature and/or moisture sensors configured to measure skin temperature and/or moisture due to sweat. These sensors may provide a signal to the sensor interface 666 indicative of the skin temperature and/or the skin moisture. The skin temperature sensor may be a contact temperature sensor (e.g., a probe in contact with the skin) or a non-contact temperature sensor. For example, the non-contact temperature sensor may be an infrared thermometer. The infrared thermometer may include a lens configured to focus infrared thermal radiation from the skin on to a detector. The detector may convert radiant power of the thermal radiation to an electrical signal. The infrared thermometer may display this electrical signal in units of temperature. Additionally, the infrared thermometer may adjust the temperature units to account for ambient temperature. The skin moisture sensor may provide Galvanic Skin Response (GSR) and impedance measurements. The sensor interface 666 may also receive blood pressure information from the cuff 160. The sensor interface may provide these signals and blood pressure information to the system controller 120 and/or the processor 570. Based on this signal, the pneumatics controller 640 may control the gas flow regulator 636 and the pressure sensor 646 to provide gas flow to the gas delivery tube 675 and the cooling tubes 690. The components 636 and 646 function substantially as described above in reference to FIG. 5 with regard to the components 530 and 540.

In an implementation, the cooling tubes 690 may include a cooling tube connection port 695. Further, the garment bladders 680 may include a garment bladder connection port 685. The connection ports 685 and 695 allow air to enter the garment bladder (e.g., during inflation) or cooling tube. Similarly, the connection port 685 allows air to exit the garment bladder during deflation. These port may include engagement features to facilitate a connection to the pneumatics controller 640, such as by an air hose. These features may include threads, clips, and the like. The connection ports 685 and 695 removably connects the garment bladders and the cooling tubes to the pneumatic distribution system. These ports may include a valve configured to disable/enable gas flow to the garment bladder 680 and/or cooling tube 690.

In an implementation, the pneumatics controller 640 may include one gas pressure source 520 coupled to the pneumatic distribution system (e.g., as represented by gas flow tubes 170, 670, and 675). Alternatively, the pneumatics controller 640 may include one or more additional gas pressure sources 620. The one or more additional gas pressure sources 620 are substantially as described above in reference to FIG. 5 with regard to the gas pressure source 520. As represented by the arrows 698 and 699, the gas pressure source 520 may provide gas flow to the cuff 160 and the gas pressure source 620 may provide gas flow to the garment bladders and/or cooling tubes.

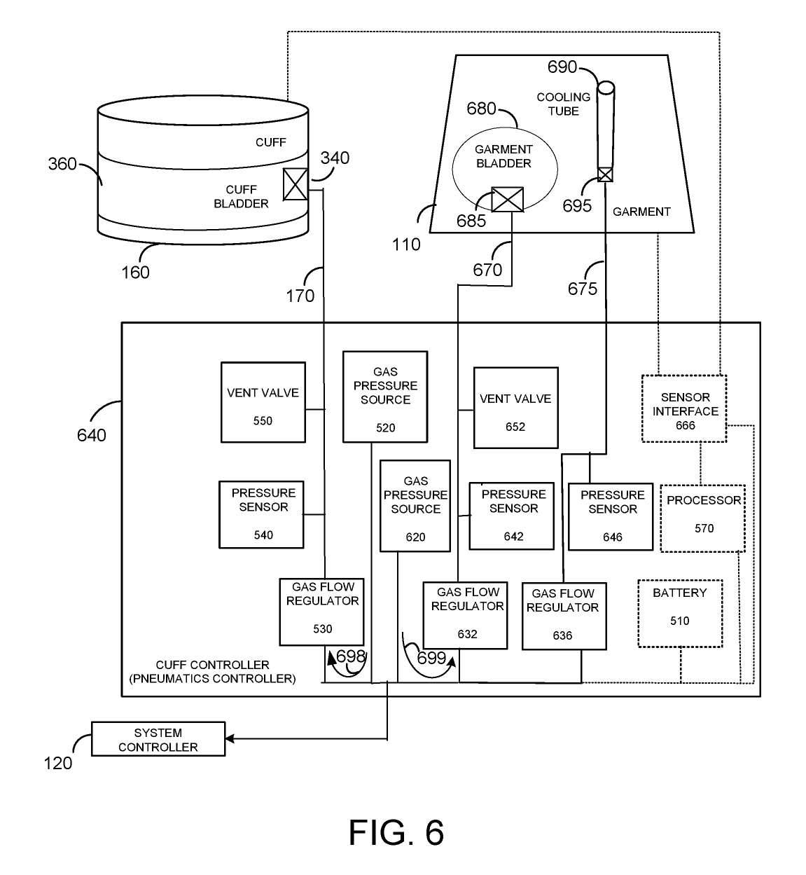

Referring to FIG. 7, a block diagram of a RIC cycle is shown. As shown in FIG. 7, a RIC treatment cycle 700 includes a cuff actuation phase 710, an ischemic phase 720, a cuff release phase 730, and a reperfusion phase 740. Optionally the RIC treatment cycle 700 includes a systolic pressure identification phase 750.

The cuff actuation phase 710 includes contracting the cuff 160 about the limb of the patient to a target set point pressure to occlude blood flow through the limb. Attainment of the target set point may be sensed through any of the herein described sensors and inflation techniques. In an implementation, the target set point may be a fixed pressure amount over the patient's systolic blood pressure. For example, the target set point may be 1-40 mmHg above the systolic pressure of the patient. In another implementation, the target set point may be a percentage of the patient's systolic blood pressure. For example, the target set point may be 101-120% of the patient's systolic blood pressure. The target set point may depend upon several factors including, but not limited to the size of the patient, the size of the patient's limb, the patient's blood pressure, confirmation of blood flow cessation, and the like.

In an implementation, the target set point may be below systolic pressure. For example, the target set point may be 85%-99% of the systolic pressure, provided that blood flow is occluded at such pressure, as may be achieved by varying cuff parameters. The pressure needed for occluding the limb and reducing most or all blood flow through the limb depends on a number of physiological factors and does not necessarily have to be greater than the systolic blood pressure of the patient. Limb ischemia sufficient for ischemic preconditioning purposes is produced when the blood flow through the limb is at least substantially reduced or preferably entirely stopped. A reduction of about 90% or greater is believed to be sufficient to cause therapeutic effects of RIC.

The ischemic phase 720 includes the controller providing control signals to the cuff controller 140 that cause the cuff 160 to remain contracted about the limb of the patient at the set point pressure for an ischemic phase duration. Maintenance of the target set point pressure may prevent reperfusion of blood flow through the limb during the ischemic phase. For example, the ischemic phase duration may be 0.5-30 minutes.

Relaxation of muscles in the patient's limb, stretching of the cuff about the limb, air leaks (intentional or unintentional), etc. may cause the cuff to relax relative to the patient's limb during the ischemic phase. Alternatively or additionally, the presence of Korotkoff sounds during the ischemic phase may indicate the onset of reperfusion. Based on pressure measurements from the pressure sensor 540 and/or the blood pressure sensors associated with the cuff 160, the system controller 120 may provide control signals to the cuff controller 140 to adjust the cuff pressure to counteract the pressure reduction.

In an implementation, the wearable system 100 may further include a blood flow sensor configured to mount on the finger or other body part of the patient and to detect the presence or absence of blood flow. The system controller 120 may receive feedback signals from the blood flow sensor and may instruct the cuff controller 140 to adjust the cuff pressure accordingly. For example, the blood flow sensor may be a pulse oximeter that may be positioned on a distal portion of the limb that receives the cuff 160, such as on a finger or toe of the limb. The pulse oximeter can provide information to the system controller 120 regarding blood pulsing through the patient's blood vessels and the percentage of hemoglobin that is saturated with oxygen. The pulse oximeter will detect an absence of pulses when blood flow though a limb is not occurring to confirm the occlusion of blood flow. Moreover, the pulse oximeter may also detect the percentage of hemoglobin saturated with oxygen, which will drop as blood flow through the limb ceases. It is to be appreciated that other sensors may also be used to confirm the cessation of blood flow, such as, for example, but not limited to, a photoplethysmographic transducer, an ultrasonic flow transducer, a temperature transducer, an infrared detector, and/or a near infrared transducer.

The cuff release phase 730 occurs at the end of the ischemic phase duration and includes the system controller 120 providing control signals to the cuff controller 140 that cause the cuff 160 to relax to allow blood flow through the limb (i.e., reperfusion). The cuff controller 140 may cause a reduction in cuff pressure to a point below diastolic pressure of the patient. In an implementation, the cuff controller 140 may cause the vent valve 550 to fully open to allow a rapid reduction in cuff pressure and a corresponding rapid relaxation of the cuff 160 about the patient's limb. In another implementation, the cuff controller 140 may control the vent valve 550 to gradually open the vent valve 550. Additionally, the cuff release may be accompanied by monitoring for the onset of Korotkoff sounds or vibrations to identify or confirm the systolic pressure of the patient. For example, the cuff 160 may be coupled to a sphygmomanometer configured for automated or semi-automated detection of Korotkoff sounds. In an implementation, the automated or semi-automated sphygmomanometer may detect Korotkoff sounds via techniques known to those skilled in the art, such as described by Geddes, et al, in “The Efficient Detection of Korotkoff Sounds”, Med. & Biol. Eng. Vol. 6, pp. 603-609. Pergamon Press, 1968.

The reperfusion phase 740 includes the system controller 120 providing control signals to the cuff controller 140 that cause the cuff 160 to remain relaxed to allow the blood flow through the limb for a time period referred to as a reperfusion phase duration. Much like the ischemic phase duration, the reperfusion phase duration may be various lengths of time, for example, 5-60 seconds, 1-30 minutes, or even longer.

To implement the systolic pressure identification phase 750, the system controller 120 may send a control signal to the cuff controller 140 to loosen the cuff 160 about the patient's limb, from a point believed to be above systolic pressure, in a systematic manner while blood pressure sensors monitor the limb for the onset of Korotkoff sounds or vibrations. Once the system controller 120 receives a feedback signal from the blood pressure sensors indicative of the systolic pressure, the system controller 120 may send a control signal to the cuff controller 140 to resume the RIC cycle.

The systolic pressure identification phase 750 may optionally occur during another phase of the RIC cycle and/or between phases of the RIC cycle and/or during selected cycles of a series of sequential cycles. For example, each cycle may begin with the identification of the patient's systolic blood pressure. In another example, the systolic pressure identification phase 750 may occur only once during an initial portion of the cycle or once during an initial portion of a first cycle of a series of sequential cycles. In a further example, the systolic pressure identification phase 750 occurs during the cuff release portion of each cycle or selected cycles in the series of sequential cycles. In an implementation, the RIC cycle may not include the systolic pressure identification phase 750 at all.

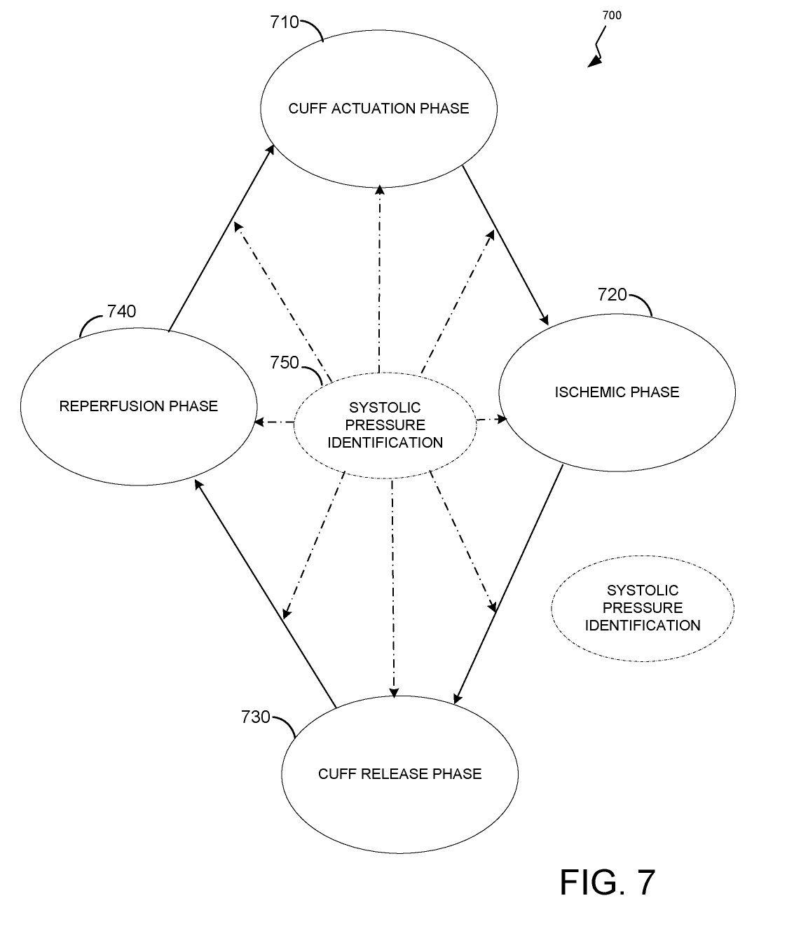

In order to provide RIC therapy to the patient, the wearable system 100 may implement a RIC treatment protocol. The RIC treatment protocol includes parameters for the various phases of the RIC cycle along with a schedule for providing the RIC therapy (i.e., a RIC therapy schedule). The system controller 120 and the cuff controller 140 are configured to implement the RIC treatment protocol by controlling the inflation and deflation or mechanical tensioning of the cuff 160. As discussed in further detail below in reference to FIG. 8, the memory 420 may include one or more predetermined RIC protocols implementable by the system controller 120 and the cuff controller 140. Alternatively or additionally, the processor 418 may be configured to determine one or more RIC protocols in real-time and store these RIC protocols in the memory 420 for implementation by the system controller 120 and the cuff controller 140. For example, the RIC protocol may include, a duration for each phase of the cycle 700, a number of times to repeat the cycle 700, and a time interval at which to implement one or more of the RIC treatment cycle 700. The time interval may be, for example, a number of times per week (e.g., 1-7 times per week), a number of times per day (e.g., 1-5 times per day), or combinations thereof (e.g., 1-5 times per day on 1-7 days of the week). In implementation, the system controller 120 may include a clock and the RIC protocol may specify specific clock times and/or days of the week in addition to or instead of intervals. For example, the RIC protocol may provide instructions to repeat the cycle 700 5 times, twice a day, for two weeks. As another example, the RIC protocol may specific 4 cycles at each of 9 a.m., 2 p.m., and 7 p.m. on Monday, Wednesday, and Friday for a month. These are illustrative examples only and other combinations of cycle numbers, time intervals, clock times, and/or days are consistent with the disclosure. In some implementations, the duration of a given phase varies from cycle to cycle within the RIC protocol. In other implementations, the duration of the given phase remains substantially constant from cycle to cycle within the same treatment. In an implementation, the RIC protocol may specify different parameters, such as different ischemic durations, reperfusion durations, pressure set points during the ischemic duration, and the like for sequential RIC cycles.

In an implementation, the RIC protocol may be based on a cardiac event risk score. The cardiac event risk score is an estimate of the likelihood or probability of the patient experiencing a future medical event if treatment efforts are not taken or not successful. Additionally, the cardiac event risk score may indicate that a series of events has begun that will likely lead to a medical event if medical intervention is not provided. As described in detail below with reference to FIGS. 8-12, the processor 418 and/or the one or more external computing devices 190 may calculate the cardiac event risk score based on physiological parameters monitored by the sensors 112 and/or 116. Unlike weather, for example, which cannot be altered substantially based on a weather prediction, the purpose for determining the cardiac event risk score is to alter the predicted course of a patient's status. The cardiac event risk score may inform and enable the wearable system 100, and/or inform and enable another device or medical personnel, to provide a course altering medical intervention. The implemented medical intervention may reduce or prevent the predicted cardiac event. Thus, the patient is able do more than just “carry an umbrella” to deal with events as they occur. For example, if a 95% chance of an adverse cardiac event is predicted within a ten-minute timeframe from the present, the wearable system 100 may alert the patient to sit down, to refrain from strenuous activity, and/or to keep the wearable system 100 on so that the wearable system 100 can provide the RIC therapy and possibly a defibrillation shock to the patient. In contrast, if the wearable system 100 determines that the patient is exhibiting an elevated level of risk of an adverse cardiac event occurring in a one-week time frame from the present, the wearable system 100 may alert a medical professional or request that the patient carefully follow the instructions of the medical professional 108.

In some cases, the cardiac event may be a myocardial infarction, otherwise known as a heart attack. In other cases, the cardiac event may be a cardiac arrest. A cardiac arrest and heart attack are two differing clinical phenomena. The heart is a muscle (referred to medically as the myocardium) and, like all muscles, it requires an oxygen-rich blood supply to function properly. The oxygen-rich blood supply is provided to the heart by coronary arteries. A heart attack occurs when there is a blockage of the coronary arteries. The heart attack is referred to medically as a myocardial infarction. Such a blockage, if not quickly resolved, can cause a portion of the heart muscle to become damaged or die. In a cardiac arrest, a change or a disturbance to the electrical properties of the heart impairs the ability of the heart to effectively pump blood. For example, the heart attack (i.e., the myocardial infarction) may damage the heart muscle. This damage may be a precursor to the cardiac arrest as this damage may cause the disturbance to the electrical properties of the heart that in turn changes the contractile properties of the heart. In accordance with aspect of the present disclosure, predicting an elevated risk of a future heart attack may trigger the system to provide an appropriate RIC protocol to the patient in advance of the future heart attack (more appropriately termed Automated Remote Ischemic Pre-Conditioning, ARIPC). Such advance RIC treatment may reduce the extent of the injury incurred as a result of the future (or potential) heart attack compared to the reduction in the extent of the injury provided by a RIC treatment after the heart attack.

Further the wearable system 100 may implement RIC therapy to treat the patient at the earliest stages of the cardiac event, well before defibrillation would be medically indicated. For example, the early application of RIC (i.e., ARIPC) prior to the onset of a heart attack may reduce the damage to the electrical properties of the heart caused by the heart attack enough to prevent subsequent cardiac arrest. Thus, such early intervention with RIC therapy may advantageously increase the chance the patient may avoid needing defibrillation and/or other aggressive therapies.

The cardiac event risk score corresponds to a time interval (e.g., a time-until-event) and a particular cardiac event. The time-until-event (TuE) (e.g., seconds, minutes, hours, days, weeks, etc.) indicates a time between the calculation of the cardiac event risk score by the controller 120 and/or the reception of the cardiac event risk score by the controller 120 and the onset of the particular cardiac event. As an example, the TuE may take the form of a vector or risk probabilities calculated for two or more times intervals (e.g. 52% risk probability at 3 days, 67% probability at 3 hours, 89% probability at 10 minutes). Examples of cardiac events include, but are not limited to, bradycardia, asystole, heart murmurs, valvular sounds such as S3 or S4 sounds, ventricular tachycardia, ventricular fibrillation, heart block, acute decompensated heart failure, reduced contractility, reduced ejection fraction, etc.

Additionally, the patient may engage in a severity elevated activity (SEA). The SEA is an activity for which the severity of injury or damage due to a cardiac event that occurs during the SEA is higher as compared to the same cardiac event that occurs without the SEA. Examples of SEAs include, but are not limited to, driving a car or other vehicle, using power tools, and holding a small child. In some cases, the SEA might include an interval during which the wearable device is either not worn by the patient, or is otherwise out of commission. For example, the patient may not wear the wearable device during swimming, bathing, and laundering of the wearable device. As another example, the wearable device may be temporarily out of commission after a previous defibrillation because defibrillation electrode electrical contact to the skin may be impaired by the previous deployment of the gel. Patient information may indicate a time at which a particular SEA may occur (e.g., a shower without the wearable system 100, strenuous exercise, air travel, laundering of the garment 110, etc.). The time between the indication of the SEA by the patient and the occurrence of the SEA is referred to herein as a time-until-activity (TuA).

As shown in the examples in Table 1 below, the RIC protocol may depend on one or more of the TuE, the TuA, the type of cardiac event, the type of SEA, and the risk probabilities associated with the cardiac event and/or the SEA. In the example below, the cardiac event is the heart attack (i.e., the myocardial infarction). A similar table may apply to other cardiac events such as, for example, cardiac arrest. Table 1 provides illustrative examples only as other time periods, events, and RIC protocols are consistent with the disclosure.

TABLE 1
Cardiac Event: Myocardial Infarction

| | Severity | | Risk | |
|---|---|---|---|---|
| Time until Event | Elevated | Time until | Probability | |
| (TuE) | Activity (SEA) | Activity (TuA) | Threshold (%) | RIC PROTOCOL |
| <10 MINUTES | Launder | TuA ≤ 60 min | 23 | 6 cycles every 1 hour |
| | garment | or on-going | | |
| 1 HOUR | Launder | TuA ≤ 60 min | 35 | 4 cycles every 1 hour |
| | garment | or on-going | | |
| 3 HOURS | Launder | TuA ≤ 60 min | 45 | 10 cycles every 3 |
| | garment | or on-going | | hours |
| 1 DAY | Launder | TuA ≤ 60 min | 55 | 12 cycles every 24 |
| | garment | or on-going | | hours |
| 1 WEEK | Launder | TuA ≤ 60 min | 65 | 2 cycles every 8 |
| | garment | or on-going | | hours |
| 1 MONTH | Launder | TuA ≤ 60 min | 85 | 3 cycles every 8 |
| | garment | or on-going | | hours |
| <10 MINUTES | Shower | TuA ≤ 60 min | 23 | 4 cycles every 2 |
| | | or on-going | | hours |
| 1 HOUR | ″ | TuA ≤ 60 min | 35 | 3 cycles every 2 |
| | | or on-going | | hours |
| 3 HOURS | ″ | TuA ≤ 60 min | 45 | 2 cycles every 3 |
| | | or on-going | | hours |
| 1 DAY | ″ | TuA ≤ 60 min | 55 | 2 cycles every 4 |
| | | or on-going | | hours |
| 1 WEEK | ″ | TuA ≤ 60 min | 65 | 2 cycles every 8 |
| | | or on-going | | hours |
| 1 MONTH | ″ | TuA ≤ 60 min | 85 | 3 cycles every 8 |
| | | or on-going | | hours |
| <10 MINUTES | Exercise | TuA ≤ 60 min | 23 | 6 cycles every 1 hour |
| | | or on-going | | |
| 1 HOUR | ″ | TuA ≤ 60 min | 35 | 2 cycles every 4 |
| | | or on-going | | hours |
| 3 HOURS | ″ | TuA ≤ 60 min | 45 | 8 cycles every 24 |
| | | or on-going | | hours |
| 1 DAY | ″ | TuA ≤ 60 min | 55 | 3 cycles every 8 |
| | | or on-going | | hours |
| 1 WEEK | ″ | TuA ≤ 60 min | 65 | 2 cycles every 8 |
| | | or on-going | | hours |
| 1 MONTH | ″ | TuA ≤ 60 min | 85 | 4 cycles every 8 |
| | | or on-going | | hours |
| <10 MINUTES | Launder | TuA > 60 min | 23 | 5 cycles every 1 hour |
| | garment | | | |
| 1 HOUR | Launder | ″ | 35 | 3 cycles every 1 hour |
| | garment | | | |
| 3 HOURS | Launder | ″ | 45 | 9 cycles every 3 |
| | garment | | | hours |
| 1 DAY | Launder | ″ | 55 | 11 cycles every 24 |
| | garment | | | hours |
| 1 WEEK | Launder | ″ | 65 | 1 cycles every 8 |
| | garment | | | hours |
| 1 MONTH | Launder | ″ | 85 | 2 cycles every 8 |
| | garment | | | hours |
| <10 MINUTES | Shower | ″ | 23 | 3 cycles every 2 |
| | | | | hours |
| 1 HOUR | ″ | ″ | 35 | 2 cycles every 2 |
| | | | | hours |
| 3 HOURS | ″ | ″ | 45 | 1 cycles every 3 |
| | | | | hours |
| 1 DAY | ″ | ″ | 55 | 1 cycles every 4 |
| | | | | hours |
| 1 WEEK | ″ | ″ | 65 | 2 cycles every 8 |
| | | | | hours |
| 1 MONTH | ″ | ″ | 85 | 2 cycles every 8 |
| | | | | hours |
| <10 MINUTES | Exercise | ″ | 23 | 6 cycles every 1 hour |
| 1 HOUR | ″ | ″ | 35 | 2 cycles every 4 |
| | | | | hours |
| 3 HOURS | ″ | ″ | 45 | 7 cycles every 24 |
| | | | | hours |
| 1 DAY | ″ | ″ | 55 | 3 cycles every 8 |
| | | | | hours |
| 1 WEEK | ″ | ″ | 65 | 2 cycles every 8 |
| | | | | hours |
| 1 MONTH | ″ | ″ | 85 | 3 cycles every 8 |
| | | | | hours |

In an implementation, the system controller 120 and the cuff controller 140 are configured to control and/or provide and receive signals to and from treatment accessories to the cuff 160 in order to implement the RIC protocol. For example, the treatment accessories may include blood pressure measurement equipment, blood pressure sensors, blood flow sensors. In such an implementation, the RIC protocol may include instructions to provide one or more systolic pressure identification phases 750 to identify the patient's systolic blood pressure. These instructions may indicate one or more points within the RIC cycle and/or within a series of sequential RIC cycles at which to implement the systolic pressure identification phase 750.

Referring to FIG. 8, a method of providing cardiac and RIC therapy to a patient is shown. The method 800 is, however, an example only and not limiting. The method 800 can be altered, e.g., by having stages added, removed, rearranged, combined, and/or performed concurrently.

At stage 810, the method 800 includes receiving one or more sensor signals indicative of cardiac information for a patient. For example, the processor 418 is configured to receive the one or more signals indicative of the cardiac information from the one or more sensing electrodes 112 and/or the additional physiological sensors 116 as described above with reference to FIG. 1. Optionally, the processor 418 may receive patient information (e.g., medical record information including age, gender, weight, body mass index, family history of heart disease, cardiac diagnosis, co-morbidity, left ventricular ejection fraction, medications, previous medical treatments, and/or other physiological information) via the user interface 150 and/or the communications network interface 406. The patient information may further include patient activity information. For example, the patient activity information may include schedules or other indications of current or upcoming exercise, showers, strenuous activities, sleeping, travel, laundering of the garment 110, etc.

At stage 820, the method 800 includes determining the cardiac information for the patient from the one or more sensor signals. For example, the processor 418 may determine the cardiac information from the one or more sensors. The cardiac information may include ECG information, EEG information, chest impedance information, blood pressure information, heart rate information, change in heart rate information, pulse oxygen information, respiration rate information, heart sound information, lung sound information, respiration sound information, tissue fluid level information, tidal CO₂ information, SpO₂ information, SMO₂ information, cerebral blood flow information, brain oxygen level information, tissue pH information, information indicating a reaction of the patient's heart to tilting of the patient, and/or ultrasound image information from sensor signals indicative of this information.

In an implementation, the processor 418 may determine the cardiac event risk score associated with a potential adverse cardiac event of the patient based at least in part on the determined cardiac information of the patient. The processor 418 may determine and/or implement the therapeutic response based on the determined cardiac event risk score, the patient information, or a combination thereof.

In an implementation, the processor 418 may determine a modified early warning score (MEWS) for the patient based on the cardiac information. MEWS is indicative of the health of the patient. MEWS is based, for example, on the systolic blood pressure, the heart rate, the respiratory rate, the temperature, and a relative responsiveness of the patient. The processor 418 may determine a partial score for each of these indicators and the sum of the partial scores may be indicative of the overall health of the patient. For example, a systolic blood pressure of <71 mmHg is assigned a +3, 71-80 mmHg is assigned a +2, 81-100 mmHg is assigned a +1, 101-199 mmHg is assigned 0, and >199 mmHg is assigned a +2. Similar scoring is applied to the other indicators. The processor 418 may evaluate the responsiveness of the patient based on a requested input to the user interface 150.

At stage 830, the method 800 includes determining if a predetermined RIC protocol is available. For example, the processor 418 may read the memory 420 to determine if the predetermined RIC protocol is stored in the memory 420. The memory 420 may include one or more stored predetermined RIC protocols. For example, a doctor, medical provider, medical equipment provider, and/or medical equipment manufacturer may download, program, input or otherwise provision the memory 420 with the stored predetermined RIC protocols.

At stage 830, the method includes selecting a predetermined RIC protocol. For example, the processor 418 may select the RIC protocol based on a selection algorithm. The selection algorithm may be based on the cardiac information, the patient information, MEWS score, the cardiac event risk score, the type of predicted event, the time-until-event, or combinations thereof. For example, the memory 420 may include a first, less aggressive RIC protocol (e.g., two RIC cycles per day on one day of the week for two months). The memory 420 may also include a second, more aggressive RIC protocol providing (e.g., four RIC cycles, four times per day for two days). In this example, the first RIC protocol provides less frequent RIC therapy than the second RIC protocol. If the patient has a high cardiac event risk score and/or has an upcoming strenuous activity, the processor 418 may select the second, more aggressive RIC protocol. Conversely, if the patient has a low cardiac event risk score and/or does not have any upcoming strenuous activity, the processor may select the first, less aggressive RIC protocol. In general, a more aggressive RIC protocol includes higher numbers of repeated RIC cycles. These RIC protocols and selection criteria are illustrative examples only and not limiting of the disclosure. In this manner, the processor 418 may select the predetermined RIC protocol that provides the RIC therapy appropriate for the current status of the patient and/or the future status of the patient. RIC therapy prior to an unmonitored activity (e.g., bathing without the wearable system 100) and/or risky activity (e.g., exercise) may provide protection from adverse medical outcomes due to these activities. In an implementation, the selection algorithm is configured to determine that none of the one or more predetermined RIC protocols are appropriate for the patient. In a further implementation, the selection algorithm is configured to select the RIC protocol based input to the user interface 150.

In an implementation, the processor 418 may select the RIC protocol based on a combination of indicators. For example, the processor 418 may evaluate a combination of cardiac indicators and patient information to determine whether or not to implement the RIC protocol. If the cardiac information indicates a rise in blood pressure and/or heart rate prior to an upcoming exercise event, the processor 418 may select a more aggressive RIC therapy protocol than if the rise in blood pressure and/or heart rate occurs without any upcoming exercise event.

In an implementation, the processor 418 may receive and/or determine a RIC protocol as part of a health evaluation procedure for the wearable system 100. The health evaluation procedure may provide patient information to the controller 120 so that the controller may tailor the monitoring and therapy to the particular needs of the patient. For example, the patient may provide information to a health survey through the user interface 150. A prescriber of the wearable system 100 may review the health survey information and determine the RIC protocol. The prescriber may download or otherwise store the RIC protocol at the controller 120. Alternatively, the processor 418 may determine the RIC protocol based on the health survey responses. The health survey may include information relevant to an evaluation of the health of the patient (e.g., sleep habits, weight, activities, stress level, etc.).

As another example of a health evaluation procedure, the patient may participate in a timed physical activity such as, for example, a walking test (e.g., a specified walking pace, duration, and distance). The wearable system 100 may monitor the physiological parameters of the patient (e.g., heart rate, pulse, ECG, etc.) and/or the patient may provide information to the user interface 150 (e.g., fatigue level, shortness of breath, etc.) before, during, and/or after the timed physical activity. The prescriber and/or the processor 418 may determine and store the RIC protocol at the controller 120 based on the information gathered during this test. The number of RIC cycles, the frequency of the RIC cycles, and/or the duration of particular phases of the RIC cycles may depend on the evaluated health of the patient. For example, the RIC protocol may include more RIC cycles and at a higher frequency for a high fatigue level and shortness of breath.

At stage 850, the method 800 includes determining a RIC protocol in real-time. For example, the processor 418 may determine the RIC protocol in real-time. The processor 418 may determine the RIC protocol in real-time if the memory 420 does not include the predetermined RIC protocol and/or if the selection algorithm determines that none of the stored predetermined RIC protocols are appropriate for the patient. The processor 418 may determine the RIC protocol in real-time in response to and based on one or more of the cardiac information, the patient information, MEWS score, the cardiac event risk score, the predicted cardiac event, and the time-until-event. For example, the processor 418 may calculate and/or determine one or more of target cuff pressure set points, RIC cycle phase durations, systolic pressure identification, and the RIC therapy schedule.

In an implementation, determining the RIC protocol in real-time may include changing a predetermined RIC protocol based on input to the user interface 150. For example, the manufacturer or distributor of the wearable system 100 may provision the wearable system 100 with one or more stored RIC protocols. The patient or a medical professional or other caregiver may change and/or override programmed settings of the stored RIC protocols. In an implementation, the user interface 150 may include security features to prevent tampering or accidental reprogramming. For example, the user interface 150 may require a security code and/or other security identification input to change and/or access the stored RIC protocols. Alternatively or additionally, the user interface 150 may include electronic and/or mechanical locks to prevent the tampering or accidental reprogramming.

At stage 860, the method 800 includes controlling contraction of a cuff according to the RIC protocol and based on the cardiac information of the patient. Having selected or determined the RIC protocol, the processor 418 determines whether or not to provide the RIC therapy (e.g., by implementing the RIC protocol) based on the cardiac information. The cardiac information may include, but is not limited to, ECG information. The processor 418 may evaluate the cardiac information to determine whether or not to provide the RIC therapy to the patient. For example, if the ECG information determined by the processor 418 indicates ectopic beats, sustained ventricular tachycardia (VT), or a series of QRS complexes, the controller 120 may implement RIC therapy as this ECG information is indicative of a possible upcoming cardiac event. ECG information may include, but is not limited to, heart rate, changes in heart rate, variability in heart rate, ST-segment measurements, ST changes, and/or measurements of characteristics of the ECG waveform. In an implementation, the processor 418 may evaluate respiratory information and/or blood pressure information in combination with the cardiac information in determining whether or not to provide the RIC therapy. The respiratory information may include, for example, rate, tidal volume, minute volume, lung sounds, and/or changes in these and/or other respiratory parameters. The blood pressure information may include, for example, systolic pressure, diastolic pressure, mean arterial pressure, and/or estimates of these blood pressure parameters as determined by, for example, a tonometric blood pressure estimation system and/or a pulse oximetry based blood pressure measurement system.

Contraction and release of the cuff are accomplished by the system controller 120 implementing instructions from the RIC protocol. The system controller 120 may provide control signals to the cuff controller 140 to implement these instructions. The control signals include, for example, signals to inflate the cuff 160, signals to deflate the cuff 160, signals indicative of a rate of inflation and/or deflation, signals indicative of a phase duration, signals to measure systolic pressure, etc. In an implementation, the system controller 120 may provide the processor 570 of the cuff controller 140 with the selected or determined RIC protocol. In a further implementation, a memory (not shown) of the cuff controller 140 may include a stored RIC protocol. In such implementation, the cuff controller 140 may implement the RIC protocol with limited control signals or without control signals from the system controller 120.

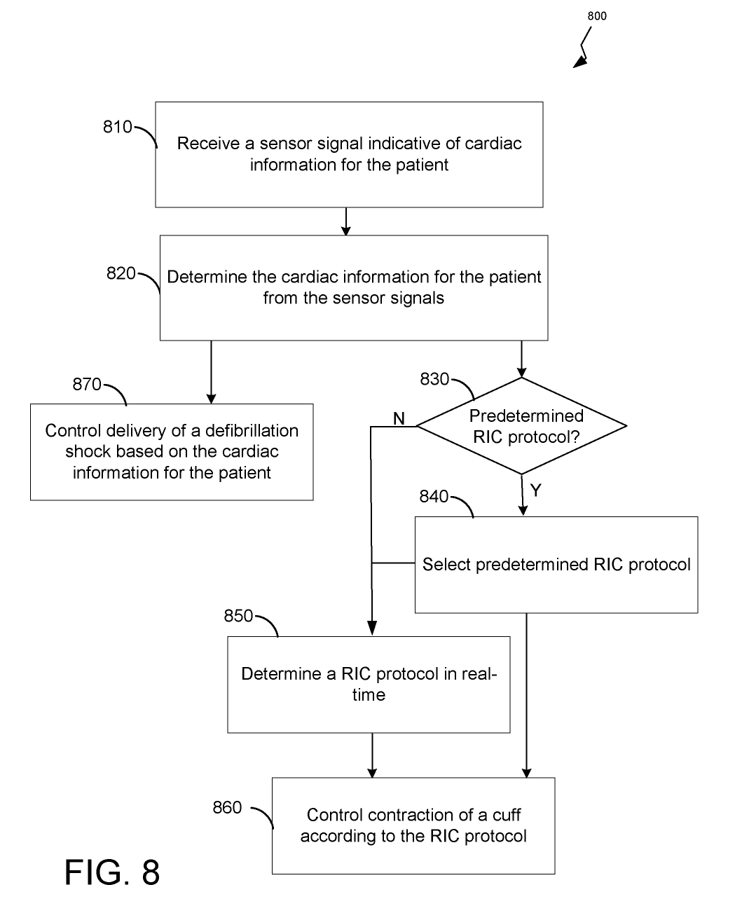

As discussed in further detail below with reference to FIG. 9, in an implementation, the processor 418 may determine whether or not to provide RIC therapy based on the cardiac event risk score determined from the cardiac information. The processor 418 may compare cardiac event risk scores for various time periods to determine a RIC therapy implementation that may vary based on the cardiac event risk scores for each of the time periods. Different time periods may be associated with different thresholds, and different medical events may be associated with different thresholds. For example, a threshold for applying defibrillation and/or RIC therapy in response to a cardiac arrest for a more immediate time period may be different, e.g., and may be easier to satisfy, than a threshold for applying defibrillation and/or RIC therapy in response to a cardiac arrest for a longer-term time period.

Alternatively or additionally, the processor 418 may provide the RIC therapy based on the MEWS and/or the patient information. For example, the processor may provide the RIC therapy for a MEWS score over a first threshold value. As another example, the processor may implement a RIC protocol with infrequent cycles for a MEWS score over the first threshold value but below a second threshold value and may implement a RIC protocol with frequent cycles for a MEWS score over the second threshold value. The higher MEWS score may correspond to a higher risk of a cardiac event.

As a further example, the patient information may include indications of current or upcoming exercise, showers, strenuous activities, sleeping, travel, laundering of the garment 110, etc. The processor 418 may cause the controller 120 to implement the RIC protocol prior to these patient activities to reduce the risk of a cardiac event during the activity. For example, in order to launder the garment 110 or shower, the patient may remove the wearable system 100. Preconditioning the vasculature of the patient with RIC therapy prior to these events may protect the patient from a cardiac event during these unmonitored periods with defibrillation and pulsing unavailable. Exercise, travel, or other activities may increase the risk of a cardiac event. RIC therapy prior to these events may reduce this risk.

The processor 418 may evaluate the time interval prior to a scheduled patient event and/or a predicted cardiac event to decide when to start the application of the RIC therapy. For example, if the patient provides input to the user interface 150 that he/she will be bathing the next day without the wearable system 100, the controller 120 may implement the RIC protocol immediately. As another example, if the cardiac event risk score indicates an expected cardiac event within the next hour or next 24 hours, the controller 120 may implement the RIC protocol immediately. Conversely, if the time interval prior to the scheduled activity is a week or more, the controller 120 may delay implementation of the RIC protocol.

Optionally, the method 800 may include storing information associated with the RIC protocol implementation. For example, the processor 418 may store information associated with the RIC protocol implementation in the memory 420. For example, the stored information the system may include cuff parameters, such as cuff pressure or tension, pneumatic controller settings and parameters, data and time information, information from feedback signals, control signal logs, and/or personal information to identify the patient. The system controller 120 may provide RIC protocol implementation information to the patient (e.g., through the user interface 150 and/or via the communications network interface 406). For example, the user interface 150 may provide audible, haptic, and/or visual indicators to accompany any of the phases of the RIC cycle. As another example, the user interface 150 may display a clock to one or more indicators of the amount of time that has elapsed or that remains for a given portion or the entire duration of the RIC cycle and/or the RIC protocol.

At stage 870, the method 800 includes controlling delivery of a defibrillation shock based on the cardiac information for the patient. For example, the processor may control the shock delivery based on the cardiac indicators determined at the stage 820 and/or based on a cardiac event risk score determined from these indicators. The cardiac event risk score is indicative of a likelihood of a cardiac event in a time period after the determination of the cardiac event risk score. For example, the cardiac event risk score may indicate a 97% chance that the patient will experience a cardiac arrest 48 hours from the time at which the cardiac event risk score is determined.

If the cardiac event risk score indicates that the patient's condition is not improving or is worsening and/or that a cardiac event is imminent, the processor 418 may implement the delivery of pulsing or defibrillation. The controller 120 may control the therapy electrodes 114 and associated circuitry to deliver the pulses or the defibrillation shock. In an implementation, the processor 418 may control an external medical device (e.g., a chest compression device, drug delivery device, etc.) to provide other therapies to the patient. The processor 418 may provide an indication of the cardiac event risk score and recommended therapy to the user interface 150. Further, the processor 418 may control the alarm manager 414 to provide alarms or reminders to the patient based on the cardiac event risk score.

In an implementation, the wearable system 100 may not include defibrillation capability. Based on the cardiac event risk score, the user interface 150 may notify the patient of an increased likelihood of an adverse event and instruct the patient reinstate the defibrillation capability of the wearable system 100 (e.g., if the defibrillation components were previously removed from the wearable system 100) or obtain another wearable system 100 that has defibrillation capability. For example, if the cardiac event risk score satisfies a switching threshold, a warning may be issued to the patient to switch from the reduced or no therapy medical device to a wearable medical device that includes therapy options.

As described above, the controller 120 may implement RIC therapy based on the cardiac event risk score as determined from physiological parameters monitored by the sensors 112 and/or 116. The monitoring capabilities of the wearable system 100 allow the wearable system 100 to pre-condition the vasculature of the patient which may minimize and/or prevent a possible and/or predicted future cardiac. Referring to FIG. 9, a method of implementing a RIC protocol in response to monitored patient parameters is shown. The method 900 is, however, an example only and not limiting. The method 900 can be altered, e.g., by having stages added, removed, rearranged, combined, and/or performed concurrently.

At the stage 910, the wearable system 100 monitors one or more parameters of a patient. These parameters include the cardiac indicators from the sensors 112 and/or 116 and/or the patient information, as described in detail above. One or more of these parameters may be predictive of an oncoming cardiac event, an oncoming vulnerability to a cardiac event (e.g., during showering, exercise, laundering the garment 110, etc.) and/or may be used to calculate the cardiac event risk score for a medical event of the patient.

At the stage 920, the processor 418 analyzes these parameters, for example, to calculate the cardiac event risk score or to determine if there is a high likelihood of an impending cardiac event and/or if a cardiac event is occurring. In an implementation, the processor 418 may send the parameters to the one or more external computing devices 190 via the communications network interface 406 for analysis and may receive the results of the analysis.

If the processor 418 determines at stage 930 that there is no cardiac event occurring and that there is not a high likelihood of an impending cardiac event, e.g., the cardiac event risk score is below a threshold, the controller 120 returns to stage 910 to monitor the parameters of the patient. In an implementation, the controller 120 may monitor different parameters of the patient at different times, for example, sequentially or in an order of priority (e.g., a priority based on an expected relevance of the parameter to cardiac events). In an implementation, the controller may monitor groups of parameters sequentially or in an order of priority.

If, at stage 930, the processor 418 determines a cardiac event is occurring or that there is a high likelihood of an impending cardiac event, the processor 418 may determine at stage 940 whether the cardiac event is treatable, for example, whether an intervention may be effective to treat the cardiac event or to reduce the chance or severity of the impending cardiac event. If the processor 418 determines that the type of cardiac event the patient is experiencing cannot currently be treated by any intervention available to the system 100 or by a first responder, the system 100 may advise at stage 950 that the patient be transported to a medical facility and the controller 120 returns to stage 910. For example, the controller 120 may activate the alarm manager 414 to provide an alarm to the patient and/or may provide information via the user interface 150. The system 100 may similarly advise the patient if no intervention available to the system 100 or to a first responder is currently capable of reducing the chance or severity of the impending cardiac event. The processor 418 may store the parameters of the patient in the memory 420 so that when the patient reaches the medical facility, medical personnel may review these parameters.

If, at stage 940, the processor 418 determines that the cardiac event that is occurring may be treated by an intervention available to the medical device or a first responder, the controller, at stage 960, makes a determination of one or more available actions (e.g., intervention) to provide to the patient. For example, the controller 120 may implement one or more of pulsing, defibrillation and/or RIC therapy. In an implementation, the controller 120 may control an external medical device (e.g., a chest compression device, drug delivery device, etc.) to provide other therapies to the patient.

At stage 970, the system 100 performs or indicates the determined intervention, for example, by administering automated RIC therapy, chest compressions, defibrillation, and/or automated drug delivery. If the selected intervention is one that the system 100 cannot perform on its own, the system 100 may provide an indication to a first responder (e.g., via the user interface 150). In an implementation, the controller 120 may communicate the recommended treatment to the one or more external computing devices 190.

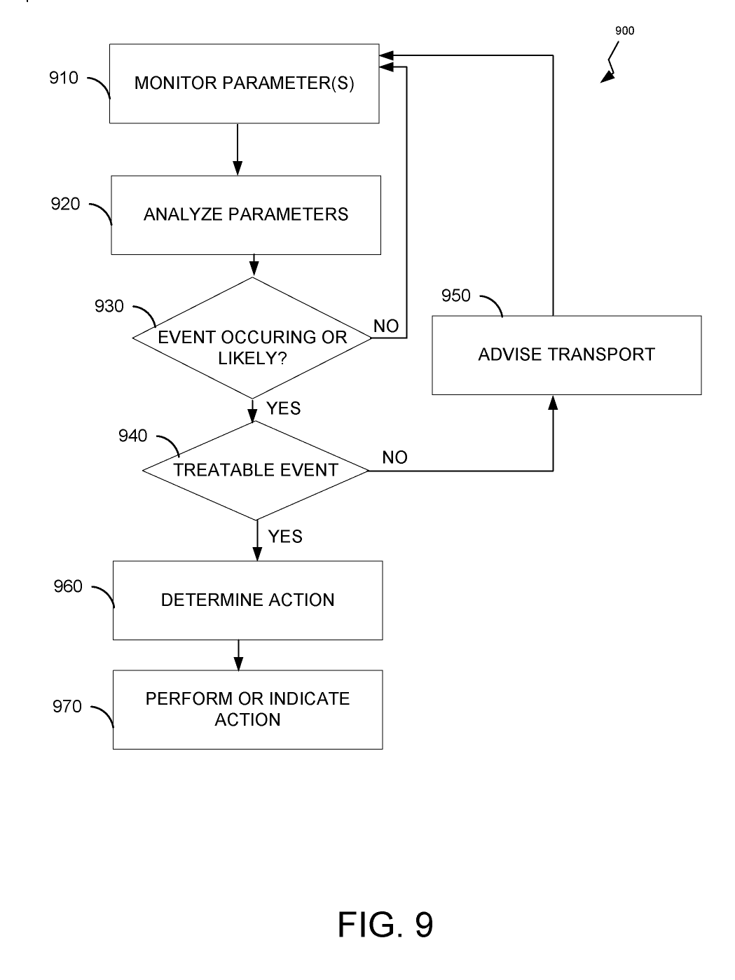

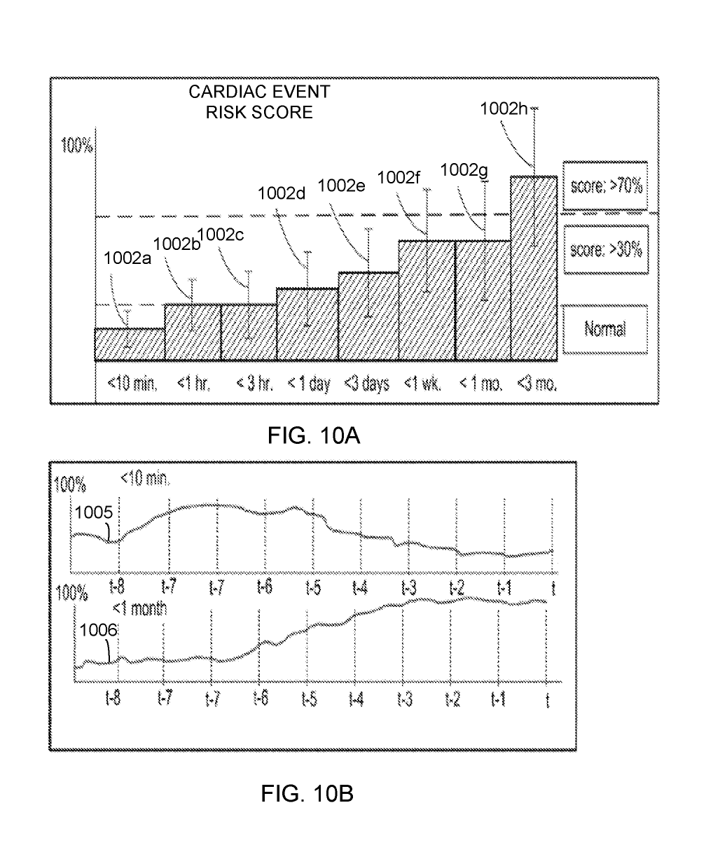

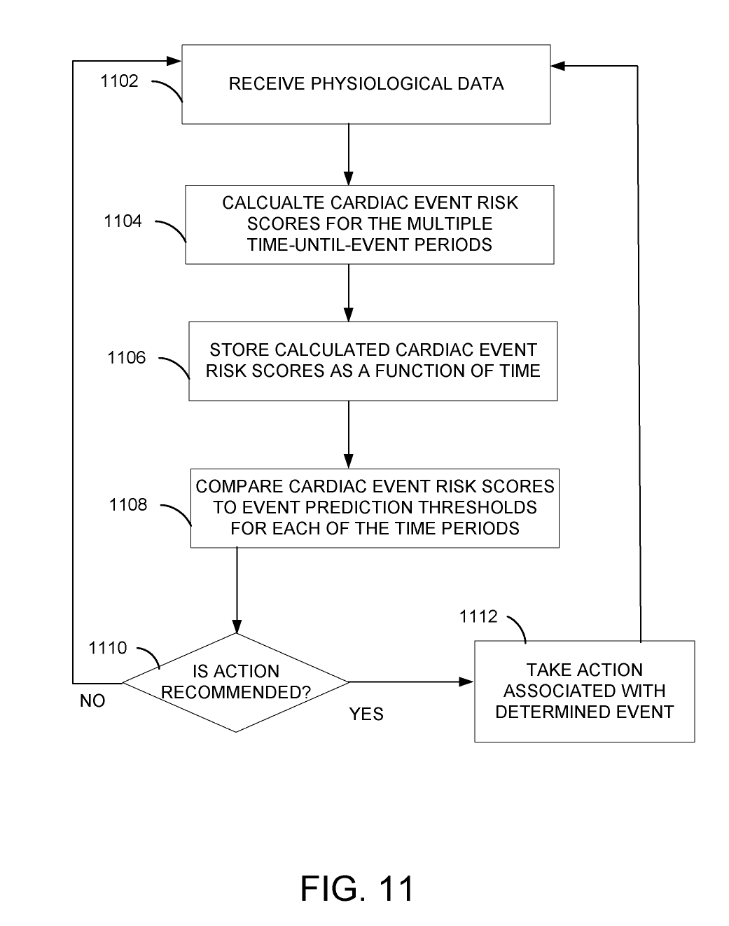

Determination of the cardiac event risk scores is now discussed in further detail. Referring to FIG. 10A, a bar graph representation of examples of cardiac event risk scores for multiple time periods is shown. The visual representations of the cardiac event risk scores for the various time periods may include information related to a reliability of the cardiac event risk score, e.g., a confidence value, associated with the corresponding cardiac event risk score. For example, a visual representation 1002a-h of the confidence value may be superimposed on each bar of the bar graph. The cardiac event risk scores may change over time. Referring to FIG. 10B, a graphical representation of examples of historical information about the progression or change in cardiac event risk scores over time is shown. The trend lines 1005 and 1006 represent trends over time in the previously calculated cardiac event risk scores. In an implementation, the controller 120 may calculate the cardiac event risk scores (e.g., as described in further detail below with regard to FIG. 11) and may determine historical information for the cardiac event risk scores as shown, for example, in FIG. 10B.

Referring to FIG. 11, a flow chart illustrating an example of a method for determining the cardiac event risk scores is shown. For example, the processor 418 may determine the cardiac event risk scores. In an implementation, the processor 418 may receive cardiac event risk scores from the one or more external computing devices 190.

At stage 1102, the controller 120 may receive, measure, and/or record one or more types of physiological data (e.g., based on one or more signals from the sensors 112 and/or 166). In an implementation, the physiological data may be previously determined data and/or may be physiological data determined by a system other than the system 100 and provided to the controller 120 (e.g., via the communications network interface 406 and/or the user interface 150). The controller 120 may further receive other patient information as described above.

At stage 1104, the controller 120 may calculate the cardiac event risk scores (e.g., estimated quantitative risk values) for multiple time periods (e.g., multiple time-until-event periods). In an implementation, the controller 120 may receive the cardiac event risk scores from the one or more external computing devices 190. The calculated cardiac event risk scores may include cardiac event risk scores corresponding to one or more time periods and/or cardiac event risk scores corresponding to one or more cardiac events. The one or more cardiac events may include multiple cardiac events of the same type (e.g., multiple cardiac arrests) and/or multiple cardiac events of different types (e.g., a combination of one or more cardiac arrests and one or more ventricular fibrillation events). The processor may further calculate an associated criticality measure and/or an associated confidence measure. For example, separate scores may be calculated and stored for time periods of before ten minutes, before one hour, before two hours, before four hours, before eight hours, before twelve hours, before twenty-four hours, before forty-eight hours, and before seventy-two hours. For each of the time periods, the processor 418 may calculate scores for multiple cardiac events (e.g., multiple cardiac events of the same kind and/or multiple cardiac events of different types).

At stage 1106, the processor 418 may store the calculated cardiac event risk scores as a function of time in the memory 420. The processor 418 may store the cardiac event risk scores for the one or more time periods and/or for the one or more medical (e.g., cardiac) events for which the score is calculated. The stored cardiac event risk scores may include the associated criticality measure and/or the associated confidence measure. In an implementation, the system 100 may provide the calculated cardiac event risk score to one or more of the user interface and/or the one or more external computing devices 190. Additionally or alternatively, the system controller 120 and/or the cuff controller 140 may determine settings and/or functions of associated hardware based on the cardiac event risk scores. For example, the settings and/or functions may include a response time for an alarm (e.g., how soon the alarm occurs after determination of the risk score), a type of alarm, a pre-charge of defibrillation capacitors (e.g., in order to reduce the time until the system is able to administer a defibrillation shock, a cuff inflation response (e.g., start cuff inflation and/or determine appropriate RIC protocol).

At stage 1108, the processor 418 may compare the cardiac event risk scores for each of the calculated time periods to event thresholds. Based on the comparison, the controller 120 may determine and implement a treatment plan. The treatment plan may include providing RIC therapy to the patient by implementing a RIC protocol.

In an implementation, the processor 418 may prioritize the cardiac event risk scores based on the criticality of each score. For example, the processor 418 may compare cardiac event risk scores having relatively higher criticality measures to stored threshold values for corresponding time periods, and determine, based on the comparison, a treatment plan or course of action before doing the same for the cardiac event risk scores having relatively lower criticality measures. In some implementations, the prioritization of the cardiac event risk scores may be based on a weighting scheme. The processor 418 may determine the weighting scheme based on one or more of the criticality measure, the confidence measure, and the risk associated with each cardiac event risk score. In some implementations, the processor 418 may prioritize cardiac event risk scores for relatively more immediate time periods above cardiac event risk scores for relatively distant time periods. Similarly, the processor 418 may prioritize multiple cardiac event risk scores for multiple different cardiac events in a same time period based on the criticality of each score or the weighting scheme.

At stage 1110, based on the results of the comparison of the cardiac event risk scores to the event estimation of risk thresholds, the controller 120 determines whether an action is recommended. For example, the controller 120 may determine that one or more of pulsing, defibrillation, chest compressions, drug delivery, and RIC therapy are recommended. Further, the controller may determine, select, or modify the RIC protocol.

If an action is recommended at stage 1110, at stage 1112, the controller 120 implements the action (e.g., the action is performed by the system 100 and/or under the direction or per the recommendation of the system 100). The method then returns to stage 1102 to receive the physiological data. If action is not recommended at stage 1110, the method returns to stage 1102 to receive the physiological data. For example, at the stage 1112, the controller 120 may send control signals to the cuff controller 140 to implement the RIC protocol.

Cardiac event risk scores may be calculated using various processes, algorithms, scoring models, mathematical models, statistical analysis, etc. A method for calculating the cardiac event risk scores may depend on a type of the medical event to be predicted. For example, the processor 418 may be configured to use a first process or algorithm to calculate the cardiac event risk score for a cardiac arrest and a second, different process or algorithm to calculate the cardiac event risk score for a ventricular fibrillation. As a further example, the processor 418 may be configured to use a first process or algorithm to calculate the cardiac event risk score for a ventricular tachycardia event or a ventricular fibrillation event and a second, different process or algorithm to calculate the cardiac event risk score for an ischemia event. A risk of impending acute degeneration of a patient's medical condition into cardiac arrest or other severe cardiopulmonary conditions may thus be calculated by a variety of methods. Different methods and algorithms may be used to calculate the cardiac event risk scores for different time periods. For example, the processor 418 may be configured to use a first process or algorithm to calculate the cardiac event risk score for a cardiac arrest in a first time period and a second, different process or algorithm to calculate the cardiac event risk score for a cardiac arrest in a second, different time period.

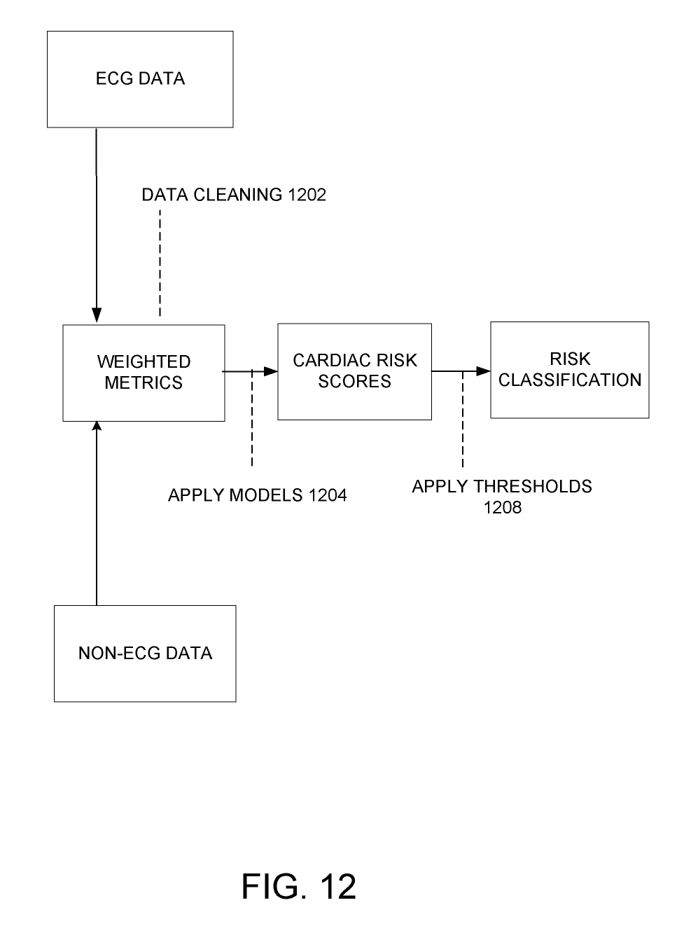

Referring to FIG. 12, a flow chart of an example of an algorithm for determining cardiac event risk scores is shown. The processor 418 may determine the cardiac event risk score based on various parameters of the patient that may be predictive of various medical events including cardiac events. As an example, the parameter may be ECG measurements. However, example implementations are not limited to ECG metrics and various other metrics can be used to calculate and classify cardiac event risk scores. For example, if the ECG measurements indicate that the QRS complex of the ECG is widening or that the heart rate of the patient is decreasing, there may be a relatively long precursor time prior to a predicted cardiac event. Further, changes to the T-wave of the patient's ECG may occur between about 15 and about 30 seconds prior to the onset of ventricular fibrillation. T-wave alternans (i.e., a periodic beat-to-beat variation in the amplitude or shape of the T-wave) may be indicative of impending ventricular tachycardia or other cardiac arrhythmia.

At stage 1202, the processor 418 (or the one or more external computing devices 190) gather and clean input data from ECG and non-ECG data sources to provide a set of weighted metrics for each patient. For example, the metrics extracted from the ECG signal may include one or more of heart rate, measurement of premature ventricular contractions, heart rate variability, PVC burden or counts, noise quantifications, atrial fibrillation, momentary pauses, heart rate turbulence, QRS height, QRS width, changes in the size or shape of the morphology, cosine R-T, artificial pacing, corrected QT interval, QT variability, T wave width, T wave alternans, T-wave variability, ST segment changes, early repolarization, late potentials, fractionated QRS/HF content, and fractionated T wave/HF content. As a further example, the metrics extracted from non-ECG data sources may include metrics indicative of patient activity. Such metrics may include patient motion metrics as monitored by an accelerometer such as, for example, a step count, a step count over a predefined time window, changes in body position during sleep or rest, etc. The processor 418 may detect fiducial points, e.g., points corresponding to P, Q, R, S, and T waves, in the ECG signal to extract individual measurements, e.g., QRS, PVC, etc., from the physiological parameter data. For example, a QT interval may provide a measure of heart failure of the patient, and the distance between the Q point and the T point may be determined and extracted from the physiological parameter signal. The metrics may further include patient information such as, for example, gender, age, and/or medical history. In particular, the metrics may include medical history related to cardiac conditions (e.g., positive indication(s) of recent myocardial infarction(s) (MI), indication(s) of MI with compromised mechanical performance, previous acute heart failure, etc.).

The processor 418 may calculate the cardiac event risk scores using various algorithms (e.g., various scoring models, mathematical models, statistical analysis, classifier models, and combinations thereof). In an implementation, the one or more external computing devices 190 may supplement the calculations performed by the processor 418. For example, the external computing devices may handle machine learning classifier models or other computations that may exceed the computational capability of the processor 418. The one or more external computing devices 190 may provide the results of computations to the processor 418. The algorithm for calculating the cardiac event risk scores may depend on a type of the cardiac event to be predicted. For example, the processor 418 may be configured to use a first algorithm to calculate the cardiac event risk score for a cardiac arrest and a second, different algorithm to calculate the cardiac event risk score for a ventricular fibrillation. Further, the processor 418 may calculate the cardiac event risk scores for different time periods using different algorithms. For example, the processor 418 may use a first algorithm to calculate the cardiac event risk score for a cardiac arrest in a first time period and a second algorithm to calculate the cardiac event risk score for a cardiac arrest in a second and different time period.

Cardiac event risk scores for individual patients can be obtained by applying machine learning algorithms to electro-physiologic data or to electro-physiologic and non-electro-physiologic data. Interpretation of the cardiac event risk scores is dependent upon threshold settings, enabling classification of cardiac event risk scores by clinically meaningful gradation of risk. Unique to each model algorithm, the threshold is determined and can be continuously refined using an available registry of patient data.

For example, at stage 1204, models such as the predictive machine learning algorithms or classifiers are applied and cardiac event risk scores are generated. The processor 418 may calculate the cardiac event risk score for one or more imminent medical events and/or for one or more longer-term medical events. In various implementations, the processor 418 may calculate the cardiac event risk score for various numbers of events and various number of time periods. For example, the processor 418 may calculate the cardiac event risk score for a single time period. In this example, processor 418 may calculate a single cardiac event risk score for a time period to provide a likelihood or probability of a medical event occurring within the time period. As another example, processor 418 may calculate a plurality of different cardiac event risk scores for a plurality of different medical events for the time period. In this example, the processor 418 may calculate the cardiac event risk score for a cardiac arrest and the cardiac event risk score for a non-sustained ventricular tachycardia for the same time period. As a further example, processor 418 may calculate cardiac event risk scores for medical events for a plurality of different time periods. In order to provide risk levels for the various different time periods, the processor 418 can calculate multiple (e.g., different) cardiac event risk scores for different time periods, (e.g., less than about 10 minutes, less than about 1 hour, less than about three hours, less than about 1 day, less than about 1 week, less than about 1 month, less than about 3 months, etc.), to provide a likelihood or probability of a medical event occurring within the respective time period.

The processor 418 may update over time the cardiac event risk score. For example, the processor 418 may initially determine the cardiac event risk score for a time period of within one hour, and as time passes, the processor 418 may update the cardiac event risk score for the time period of within one hour. Further, the processor 418 may calculate the cardiac event risk scores as a function of time. In this case, the cardiac event risk scores may predict changes in the medical condition of the patient over time.

The cardiac event risk score may include, or be associated with, a criticality measure and/or a confidence measure. The criticality measure can provide an estimated measure of the risk, severity or significance of a potential medical event. For example, a medical event, such as a cardiac arrest, may be associated with a relatively high criticality measure while a medical event, such as an ectopic beat or a non-sustained ventricular tachycardia, may be associated with a relatively lower criticality measure. The criticality measure may be a quantitative score or a qualitative assessment. The confidence measure can provide a likelihood or probability that a particular medical event may occur for the associated time period. The confidence measure may be a percent chance of event occurrence for the associated time period. For example, the confidence measure may indicate that 2% of patients presenting with particular input data to the cardiac risk score will experience a subsequent cardiac arrest.

The processor 418 may use the criticality measure and/or the confidence measure of the cardiac event risk score to determine an appropriate response to the medical event, such as an appropriate treatment and/or intervention. The treatments and/or interventions may include, for example, defibrillation, CPR, calling an ambulance, calling a physician, RIC therapy, changing and/or ceasing activity (e.g., sitting down, lying down, stopping a walk or an exercise activity), providing the patient with diagnostic information and/or an alarm. Some treatments, such as defibrillation, may require a relatively higher confidence value prior to implementation. Other treatments, such as implementing the RIC therapy, may require a relatively lower confidence prior to implementation. The risk of a degradation in the patient's health in response to RIC is low (e.g., relative to the risk associated with defibrillation). Therefore, as an example, the system 100 may implement RIC as a pre-cautionary treatment even with the confidence measure of 2% as discussed above. As a further example, defibrillation may require a relatively higher criticality measure than the RIC therapy. For instance, the defibrillation may require a criticality score associated with cardiac arrest and the RIC therapy may require a criticality score associated with an increased heart rate.

At stage 1208, thresholds (e.g., predetermined thresholds) based upon performance criteria are used to interpret the patient's cardiac event risk score by gradation of risk to classify the cardiac event risk score. In some examples, the thresholds for the classifier models can be determined by applying previously developed machine learning algorithms to historical data available in a registry of patient data with known outcomes for sudden cardiac death, including sudden cardiac arrest and asystole. Patients can be chosen by any number of criteria, such as consecutive entry into the registry, cardiac diagnosis, gender, etc. The determination of a threshold can be based upon predetermined performance criteria, such as 95% specificity or other diagnostic test performance criteria. For example, in the case of 95% specificity, the cardiac event risk scores from a group of patients with negative outcome for the test criteria can be ordered by value and the cutoff for the 95th percentile determined using standard methods. Moreover, thresholds for other performance criteria such as sensitivity, positive predictive value, number needed to diagnosis, etc. can be predetermined in a similar manner.

Thresholds can be chosen to achieve any clinically meaningful level of specificity, sensitivity, positive predictive value, number needed to diagnose or other diagnostic performance characteristic. In an example, the threshold for interpretation of cardiac event risk scores is predefined in order to achieve performance at 95% specificity. This decision determines the levels of the other performance characteristics. In an implementation, the thresholds may be based on historical normative values for groups of patients. For example, when 10,000 patients are examined and the prevalence of sudden cardiac arrest is 1.4%, the corresponding threshold classifies 493 patients as false positive and 9,367 patients as true negative. If the sensitivity performance of the models is 20%, 28 patients are classified as true positive and 112 are classified as false negative. In a similar fashion, additional performance characteristics, such as positive predictive value, negative predictive value, number need to diagnose, etc., can be specified in advance in order to achieve individual performance measures of predetermined clinical significance. In a further implementation, the thresholds may be based on baseline (e.g., historical data) values for the particular patient.

In some examples, a patient's cardiac event risk score being above the threshold can lead to a direct notification to the patient, the patient's medical team or responsible third party such as a close family member. For example, the user interface 150 may provide a notification of elevated risk to the patient. As another example, the controller 120 may send a notification of the patient's elevated risk status to the patient's medical team via the communications network interface 406.

In some examples, the cardiac event risk score can be evaluated by incorporating electro-physiologic information collected at different times. The time series data can provide renewed cardiac event risk scores at intervals that range from a few seconds or minutes to days. Use of multiple cardiac event risk scores can be optimized to follow any number of approaches to determine the patient's overall risk for sudden cardiac death. For example, a cardiac event risk score occurring above the threshold can be used to classify a patient as presenting elevated risk. Moreover, other variables can be used to evaluate risk, such as but not limited to changes in risk values (whether increasing or decreasing), the length of time a patient remains above the risk threshold, or an area under the curve based approach.

Identification of cardiac event risk scores associated with true positive patients can lead to actionable outcomes that impact the patient's health. The performance of the cardiac event risk score can be valid for a range of times spanning from seconds or minutes to days leading to different recommended actions.

Other Considerations:

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device.

A computer program is a set of instructions that can be used by a computer processor to perform some activity or bring about some result. A computer program can be written in any form of programming language, including compiled or interpreted languages, and it can be deployed in any form, including as a stand-alone program or as a module, component, subroutine, or other unit suitable for use in a computing environment. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

The system controller 120 described herein may include, or be operatively coupled to communicate with, one or more mass storage devices for storing data files; such devices include magnetic disks, such as internal hard disks and removable disks; magneto-optical disks; and optical disks.

The terms “machine-readable medium,” “computer-readable medium,” and “processor-readable medium” as used herein, refer to any medium that participates in providing data that causes a machine to operate in a specific fashion. Using a computer system, various processor-readable media (e.g., a computer program product) might be involved in providing instructions/code to processor(s) for execution and/or might be used to store and/or carry such instructions/code (e.g., as signals).

In many implementations, a processor-readable medium is a physical and/or tangible storage medium. Such a medium may take many forms, including but not limited to, non-volatile media and volatile media. Non-volatile media include, for example, optical and/or magnetic disks. Volatile media include, without limitation, dynamic memory.

Common forms of physical and/or tangible processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of processor-readable media may be involved in carrying one or more sequences of one or more instructions to one or more processors for execution. Merely by way of example, the instructions may initially be carried on a flash device, a device including persistent memory, and/or a magnetic disk and/or optical disc of a remote computer. A remote computer might load the instructions into its dynamic memory and send the instructions as signals over a transmission medium to be received and/or executed by a computer system.

The system controller 120 may be communicatively coupled to a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet. The computer system can include clients and servers. A client and server are generally remote from each other and typically interact through a network, such as the described one. The relationship of client and server arises by virtue of computer programs running on the respective computers and having a client-server relationship to each other.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Information and signals may be represented using any of a variety of different technologies and techniques. For example, data, instructions, commands, information, signals, and symbols that may be referenced throughout the above description may be represented by voltages, currents, electromagnetic waves, magnetic fields or particles, optical fields or particles, or any combination thereof.

The methods, systems, and devices discussed above are examples. Various alternative configurations may omit, substitute, or add various procedures or components as appropriate. Configurations may be described as a process which is depicted as a flow diagram or block diagram. Although each may describe the operations as a sequential process, many of the operations can be performed in parallel or concurrently. In addition, the order of the operations may be rearranged. A process may have additional stages not included in the figure. Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

Components, functional or otherwise, shown in the figures and/or discussed herein as being communicatively coupled may be coupled via one or more wired and/or wireless connections so as to enable communication between the components.

As used herein, including in the claims, “and” as used in a list of items prefaced by “at least one of” indicates a disjunctive list such that, for example, a list of “at least one of A, B, and C” means A or B or C or AB or AC or BC or ABC (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

Having described several example configurations, various modifications, alternative constructions, and equivalents may be used without departing from the disclosure. For example, the above elements may be components of a larger system, wherein other rules may take precedence over or otherwise modify the application of the invention. Also, a number of operations may be undertaken before, during, or after the above elements are considered. Also, technology evolves and, thus, many of the elements are examples and do not bound the scope of the disclosure or claims. Accordingly, the above description does not bound the scope of the claims. Further, more than one invention may be disclosed.

Other implementations are within the scope of the invention. For example, due to the nature of software, functions described above can be implemented using software, hardware, firmware, hardwiring, or combinations of any of these. Features implementing functions may also be physically located at various locations, including being distributed such that portions of functions are implemented at different physical locations.

1. A wearable medical treatment system for medical treatment of a patient, the wearable medical treatment system comprising:
a plurality of sensors configured to be externally positioned on the patient, the plurality of sensors comprising at least one cardiac sensing electrode configured to provide one or more signals indicative of cardiac information of the patient;
at least one defibrillation electrode configured to be externally positioned on the patient and to deliver a defibrillation shock to the patient;
a cuff configured to contract about a limb of the patient; and
a controller communicatively coupled to the plurality of sensors, the at least one defibrillation electrode, and the cuff, wherein the controller is configured to:
receive the one or more signals indicative of the cardiac information of the patient,
determine the cardiac information of the patient,
control delivery of the defibrillation shock by the at least one defibrillation electrode based at least in part on the cardiac information of the patient, and
control contraction of the cuff based according to a remote ischemic conditioning (RIC) protocol and based at least in part on the cardiac information of the patient.

2. The wearable medical treatment system of claim 1 wherein the cardiac information of the patient comprises information indicative of whether a cardiac arrhythmia is occurring in the patient.

3. The wearable medical treatment system of claim 1 wherein the controller is configured to:
determine a cardiac event risk score associated with a potential adverse cardiac event of the patient based at least in part on the cardiac information of the patient; and
implement the RIC protocol based on the cardiac event risk score.

4. (canceled)

5. The wearable medical treatment system of claim 1 further comprising a user interface communicatively coupled to the controller and configured to capture input from the patient wherein the controller is configured to control contraction of the cuff based on the input from the patient.

6. (canceled)

7. The wearable medical treatment system of claim 5 wherein the controller is configured to determine the RIC protocol based on the input from the patient.

8. The wearable medical treatment system of claim 1 wherein the RIC protocol includes a plurality of sequential treatment cycles, each cycle comprising:
a cuff contraction phase during which the controller causes the cuff to contract about the limb of the patient to a set point pressure above a systolic pressure of the patient to occlude blood flow through the limb;
an ischemic phase during which the controller causes the cuff to remain contracted about the limb of the patient at the set point pressure for an ischemic phase duration;
a cuff release phase during which the controller causes the cuff to relax to allow the blood flow through the limb; and
a reperfusion phase during which the controller causes the cuff to remain relaxed to allow the blood flow through the limb for a reperfusion phase duration.

9. The wearable medical treatment system of claim 8 wherein the ischemic phase duration is between 0.5 and 30 minutes.

10. The wearable medical treatment system of claim 8 wherein the reperfusion phase duration is between 0.5 and 30 minutes.

11. The wearable medical treatment system of claim 8 wherein the RIC protocol further comprises a time interval at which to implement the plurality of sequential treatment cycles.

12. The wearable medical treatment system of claim 11 wherein the time interval is a number of times per week.

13. The wearable medical treatment system of claim 12 wherein the time interval is between 1 and 7 times per week.

14. The wearable medical treatment system of claim 11 wherein the time interval is a number of times per day.

15. The wearable medical treatment system of claim 14 wherein the time interval is between 1 and 5 times per day.

16. The wearable medical treatment system of claim 1 further comprising:
a gas pressure source;
a gas flow regulator coupled to the gas pressure source and the controller; and
a pneumatic distribution system coupled to the gas flow regulator.

17. The wearable medical treatment system of claim 16 wherein the cuff comprises a pneumatically inflatable bladder removably connected to the pneumatic distribution system and further wherein the controller is configured to adjust a gas pressure in the pneumatically inflatable bladder via control of the gas flow regulator.

18. The wearable medical treatment system of claim 17 wherein the cuff comprises a connection port and the pneumatic distribution system comprises a gas flow tube configured to removably connect to the connection port.

19. The wearable medical treatment system of claim 17 wherein the pneumatically inflatable bladder is confined to a portion of the cuff configured to be proximate to a surface of an arm of the patient that approximately faces the torso of the patient.

20. The wearable medical treatment system of claim 16, further comprising a garment that is configured to be worn about the torso of the patient, wherein the pneumatic distribution system is integrated into the garment.

21. The wearable medical treatment system of claim 20 wherein at least a portion of the garment comprises pneumatically inflatable bladders connected to the pneumatic distribution system.

22. The wearable medical treatment system of claim 21 wherein the pneumatically inflatable bladders are configured to inflate and press against one or more of the plurality of sensors and the at least one defibrillation electrode.

23. The wearable medical treatment system of claim 21 wherein the plurality of sensors comprises pressure sensors integrated into the garment and configured to detect pressure from the garment on the patient and wherein the controller is configured to adjust a gas pressure in the pneumatically inflatable bladders, in response to a pressure sensor signal, via control of the gas flow regulator.

24. The wearable medical treatment system of claim 20 wherein at least a portion of the garment comprises cooling tubes connected to the pneumatic distribution system, wherein the cooling tubes are configured to release compressed gas in a direction of the torso of the patient.

25. The wearable medical treatment system of claim 24 wherein the plurality of sensors comprises moisture sensors configured to detect moisture on the skin of the patient and wherein the controller is configured to provide the compressed gas to the cooling tubes, in response to a moisture sensor signal, via control of the gas flow regulator.

26. The wearable medical treatment system of claim 16 wherein the at least one defibrillation electrode comprises an inflatable cell connected to the pneumatic distribution system and an electrolyte gel sac in contact with the inflatable cell and wherein the controller is configured to increase a gas pressure in the inflatable cell in order to release electrolyte gel from the electrolyte gel sac via control of the gas flow regulator.

27. The wearable medical treatment system of claim 1 wherein the cardiac information of the patient comprises one or more of electrocardiogram (ECG) information, pulse information, chest impedance information, and heart rate information.

28. The wearable medical treatment system of claim 1 wherein the plurality of sensors comprises one or more of a blood pressure sensor, a motion sensor, a chest impedance sensor, a pulse oxygen level sensor, and a respiration rate sensor.

29. The wearable medical treatment system of claim 1 wherein at least a portion of the cuff comprises one or more of at least one elastic strap and an adhesive surface.

30. (canceled)

31. The wearable medical treatment system of claim 1 wherein the cuff is disposable.

32. The wearable medical treatment system of claim 1 wherein an external surface of the cuff comprises a microporous fabric.

33. The wearable medical treatment system of claim 1 comprising a mobile power supply.

34. The wearable medical treatment system of claim 1 comprising a communications network interface coupled to the controller.

35. The wearable medical treatment system of claim 34 wherein the controller is configured to transmit one or more of the cardiac information, an indication of delivery of the defibrillation shock, an indication of an implementation of the RIC protocol, or combinations thereof.

36. The wearable medical treatment system of claim 34 wherein the controller is configured to receive the RIC protocol via the communications network interface.

37. The wearable medical treatment system of claim 1 wherein the controller is configured to implement the RIC protocol prior to an expected cardiac event, wherein the cardiac information is predictive of the expected cardiac event.

38.-52. (canceled)
